(19) United States
(12) Patent Application Publication
(10) Pub. No.: US 2026/0166000 A1
(43) Pub. Date: Jun. 18, 2026

(54) 1,8-CINEOLE CONTAINING COMPOSITION FOR THERAPEUTIC USE

(71) Applicant: Maria Clementine Martin Klosterfrau Vertriebsgesellschaft mbH, Köln (DE)

(72) Inventors: Michael PLOCH, Köln (DE); Ralph Pries, Lübeck (DE); Karl-Ludwig Bruchhage, Lübeck (DE)

(21) Appl. No.: 19/533,537

(22) Filed: Feb. 9, 2026

Related U.S. Application Data
(62) Division of application No. 18/258,455.

(30) Foreign Application Priority Data
Dec. 22, 2020 (DE) 102020134587.9

(51) Int. Cl. A61K 31/352; A61K 9/127; A61P 1/14; A61P 29/00

(52) U.S. Cl. CPC A61K 31/352; A61K 9/127; A61P 1/14; A61P 29/00

(57) ABSTRACT

The present invention relates to an active ingredient and a drug or composition, respectively, for use in reducing pathogenic germs in the human intestine or for use in reducing colonization of the human intestine with pathogenic germs, preferentially for the purpose of preventing, reducing or curing inflammatory diseases or preferentially for the purpose of prophylactic or therapeutic treatment of inflammatory diseases of the human body.

### CROSS-REFERENCES TO RELATED APPLICATIONS

This is a divisional application that claims priority to and the benefit of co-pending U.S. patent application Ser. No. 18/258,455, filed on Jun. 20, 2023, which is a National Stage filing of International Application PCT/EP 2021/077049 filed Oct. 1, 2021, entitled “COMPOSITION CONTAINING 1,8-CINEOLE FOR THERAPEUTIC USE” claiming priority to DE 10 2020 134 587.9 filed Dec. 22, 2020. The subject application claims priority to co-pending application Ser. No. 18/258,455, to DE 10 2020 134 587.9 and PCT/EP 2021/077049 and incorporates all by reference herein, in their entirety.

### BACKGROUND OF THE INVENTION

The present invention relates to the technical (i.e. medical-pharmaceutical) field of the microbiome of the human intestine and especially of the human intestinal flora and further of inflammatory diseases of the human body, which are especially associated with pathogenic germs in the intestinal flora or a malcolonization of the intestine or are caused or aggravated thereby.

More particularly, the present invention relates to an active ingredient and a drug or composition, respectively, for use in reducing pathogenic germs in the human intestine or for use in reducing colonization of the human intestine with pathogenic germs, preferentially for the purpose of preventing, reducing or curing inflammatory diseases or preferentially for the purpose of prophylactic or therapeutic treatment of inflammatory diseases of the human body, respectively. Similarly, the present invention also relates to an active ingredient and a drug or composition, respectively, for use in the prophylactic or therapeutic treatment of dysbiosis or malcolonization of the human intestine. Accordingly, the present invention also relates to the use of an active ingredient and a drug or composition in this regard.

According to the invention, cineole, preferentially 1,8-cineole, or a drug containing this active ingredient or a corresponding composition is used as the active ingredient.

Furthermore, the present invention also relates to a pharmaceutical combination based on the active ingredient used according to the invention in the form of cineole, preferentially 1,8-cineole.

The human digestive tract or gastrointestinal tract is generally formed by organs that serve to absorb, break down and transport food, wherein metabolic processes take place in the digestive tract that lead to the release or breakdown of nutrients contained in the food so that these are available to the body in a usable form and to enable the nutrients to be absorbed into the body. In the digestive tract, for example, the food is broken down mechanically and enzymatically, followed by the absorption of nutrients, electrolytes and water, and also the excretion of indigestible or unusable food components from the body.

The human intestine with its small and large intestines plays an important role in the breakdown and absorption of nutrients, electrolytes and water. In addition to enzymes, numerous microorganisms that colonize the intestine are also involved in digestion. The totality of microorganisms that colonize the human intestine is referred to as the intestinal flora, which represents a separate or independent human microbiome. In this context, the intestine with the underlying intestinal flora represents a complex and dynamic bacterial ecosystem, which is established within the first years of life and which develops with increasing age and is also subject to changes, which can also be of a pathological nature. The intestinal flora is characterized by the presence of a large number of different bacterial families and genera and the species on which they are based.

Undesirable changes in the intestinal flora can be caused, for example, by an improper diet, the ingestion of contaminated food and the excessive use of medications such as antibiotics, cortisone, or the like. In addition, excessive alcohol consumption can also lead to an undesirable change in the intestinal flora. Changes in the intestinal flora can be accompanied by under- or over-colonization as well as by a change in the bacterial composition, which can occur both in the area of the large intestine and in the area of the small intestine. A malcolonization of the intestine, which is associated especially with an excessive presence or excessive growth of pathogenic germs in the intestinal flora, can persist over long periods of time and thus become chronic. The imbalance of the intestinal flora resulting from a malcolonization of the intestine is generally also referred to as dysbiosis or dysbacteria, which refers to an excessive presence of harmful or pathogenic foreign flora in the intestine.

Numerous approaches to influencing the intestinal flora are known in the state of the art, with a particular focus on the treatment of acute disorders of the intestinal flora, especially with regard to alleviating or preventing diarrhea symptoms or diseases associated with acute impairments of the intestinal flora, as for example with the frequently occurring traveler's diarrhea. This is an infectious disease of the intestine caused by pathogenic bacteria or their toxins, wherein pathogenic enterotoxin-producing bacteria of the Escherichia coli (ETEC) type are involved in more than 50% of the cases that occur. The fluid loss associated with diarrhea can lead to dehydration, accompanied by significant electrolyte loss and consequent electrolyte deficiency.

In the prior art, pharmaceutical preparations that reduce or suppress intestinal activity are often used to treat diarrhea, as is the case with the active ingredient loperamide, which belongs to the opioids. The underlying effect is a reduction in intestinal peristalsis, which reduces the symptoms of diarrhea. However, this impedes or delays the excretion of pathogens or toxins. Particularly with regard to acute diarrhea associated with intestinal malcolonization, electrolytes or minerals, which may also be combined with glucose, are also administered in the prior art, especially in the form of aqueous solutions. Although this can compensate for the loss of electrolytes and fluids, it does not combat the pathogens causing the diarrhea. In addition, the administration of living or viable microorganisms is also considered in the prior art, particularly for the treatment of diarrhea. However, the efficacy of such applications is not always sufficient.

In addition, preparations based on prebiotic components and microorganisms that influence the intestinal flora are also used in the prior art with regard to diarrheal diseases associated with intestinal malcolonization. For example, DE 10 2008 059 070 A1 relates to a composition which is to be used especially for the therapeutic or prophylactic treatment of diarrheal diseases, wherein the composition in question contains microorganisms and at least one prebiotic. This is intended to support the body's own natural intestinal flora. On this basis, relatively good effects can be achieved with regard to the treatment of acute diarrhea symptoms or diseases. However, on this basis, a targeted influence or adjustment of the intestinal flora is sometimes not possible, especially regarding the aspect of improving the immune system influenced by the microbiome of the intestine or the intestinal flora or the underlying immune status and associated or related and also on a physical or systemic level existing inflammatory diseases.

In addition to the important digestive function with for example the further breakdown of nutrients, the intestinal flora and the underlying germs and bacteria are also of great importance with regard to the immune system and inflammatory processes in the human body, for example with regard to the defense against and fight against pathogens and further immune modulation and influence on inflammatory processes. The intestinal flora or the intestinal microbiome also has a high regulatory influence on inflammatory processes and the associated immune regulation. In addition to a negative influence on the digestive function, an excessive lack of pathogenic bacteria in the intestine or a dysbiosis is therefore often associated with adverse effects on the immune system or on underlying immune (defense) reactions or regulations.

It is also noteworthy that in the human body about 70% to 80% of all (immune) cells capable of producing antibodies are localized in the intestinal mucosa or in the intestinal epithelium. Such antibody-producing cells require an intact intestinal flora to function in an optimal manner. Accordingly, a sustained disturbance of the intestinal flora or dysbiosis can also have effects on such immune-relevant cells, accompanied by an underfunction or dysfunction of the immune system also with regard to inflammatory processes in the human body as a whole.

In addition, pathogenic germs or pathogenic bacteria can have properties that initiate or influence inflammatory reactions and can thus lead to corresponding inflammatory diseases or intensify or modulate inflammatory diseases, such as the production or release of corresponding inflammatory relevant substances, which are also relevant on a (total) physical level. A malcolonization of the intestine can thus lead to an influence on the immune system, accompanied by an increased susceptibility to infections or an undesirable amplification or initiation of inflammatory processes.

A malcolonization of the human intestine is all the more problematic with regard to its influence on immune or inflammation-relevant processes, as the presence of pathogenic germs in the intestine and the presence of an intestinal epithelium weakened as a result of this, for example, can intensify one another in an undesirable way with regard to their negative effect on the immune system or inflammatory processes as a whole.

The consequences of intestinal overgrowth or dysbiosis are therefore not only negative effects on digestive function as such, but also a negative influence on the immune status of a patient affected by this, also with regard to influencing or causing inflammatory processes and the associated immune regulation. A change in the immune status or the strength of the defense caused by a malcolonization of the human intestine with pathogenic germs or by dysbiosis can also have corresponding effects on the whole-body processes of a patient affected by this. Such intestinal malcolonization or dysbiosis can also lead to a higher susceptibility to infections or inflammatory diseases in general or to inflammatory reactions at the level of the whole body, also with regard to influencing or modulating inflammatory diseases or processes that are taking place or are already present in the body as a whole. As a consequence, malcolonization and dysbiosis have an effect on the human body as a whole that goes beyond the human intestine as the site of their presence, especially with regard to a regulatory influence on inflammatory processes and corresponding immunoregulatory processes, so that diseases and inflammatory processes in the body as a whole can be influenced or intensified on this basis.

### BRIEF SUMMARY OF THE INVENTION

In light of the above explanations, there is thus a great need—especially also due to the sometimes great impact of a malcolonization of the intestine with pathogenic germs or a dysbiosis on the overall state of health or the immune status—to provide suitable forms of therapy on the basis of efficient active ingredients or related compositions and drugs which are suitable in an efficient manner for reducing pathogenic germs in the human intestine or in the human intestinal flora and indeed also against the background of improving the immune status or avoiding or reducing inflammatory diseases of the human body as a whole, which are particularly associated with underlying malcolonization of the human intestine or in the human intestinal flora, also against the background of the improvement of the immune status or the avoidance or reduction of inflammatory diseases of the human body as a whole, which are present or caused or negatively influenced or intensified by pathogenic germs in the human intestine, especially in connection with the underlying malcolonization of the human intestine.

Thus, the object on which the present invention is based is to provide an efficient concept or related active ingredients and compositions as well as drugs, respectively, which, when used or applied, lead to a reduction of pathogenic germs in the human intestine or to a reduction of the colonization of the human intestine with pathogenic germs and thus to a normalization or improvement of the human intestinal flora, wherein, in this respect, a reduction or avoidance or cure of inflammatory diseases of the human body or a corresponding treatment of inflammatory diseases of the human body is also to be made possible. In addition to good efficacy, excellent tolerability and simple applicability should also be ensured at the same time.

Likewise, a further task of the present invention is to provide corresponding active ingredients as well as compositions and drugs based thereon, which are suitable especially for the prophylactic and/or therapeutic treatment of dysbiosis or malcolonization of the human intestine as such with pathogenic germs, especially also with regard to a further positive influence on inflammatory diseases of the human body as a whole, whereby also in this respect a good tolerability with simple applicability is to be given with high efficacy.

A further task of the present invention is also to provide corresponding active ingredients or compositions and drugs based thereon with regard to the reduction of pathogenic germs in the human intestine or with regard to the reduction of the colonization of the human intestine with such pathogenic germs, which at least largely avoid or at least mitigate the disadvantages of the prior art described above.

In a completely surprising way, the applicant has now found out that cineole, especially 1,8-cineole, is unexpectedly and efficiently suitable as an active ingredient for a sustainable reduction of pathogenic germs in the human intestine or for a related reduction of the colonization of the human intestine with pathogenic germs, namely also in connection with an avoidance, reduction or cure of inflammatory diseases of the human body, which especially are associated with such a malcolonization or are caused by pathogenic germs of the intestinal flora or are induced or increased thereby.

To solve the problem described above, the present invention therefore proposes—according to a first aspect of the present invention—a cineole, especially 1,8-cineole, for use in reducing pathogenic germs in the human intestine or for use in reducing the colonization of the human intestine with pathogenic germs, respectively, preferentially for the purpose of preventing, reducing or curing inflammatory diseases of the human body or preferentially for the purpose of prophylactic or therapeutic treatment of inflammatory diseases of the human body. In accordance with the present aspect, the present invention also relates to cineole, preferentially 1,8-cineole, for use in the prophylactic or therapeutic treatment of dysbiosis or of malcolonization of the human intestine with pathogenic germs.

A further object of the present invention—according to a second aspect of the present invention—is moreover the use of a cineole, preferentially 1,8-cineole, according to the present invention (for the preparation of a drug or medicament) for reducing pathogenic germs in the human intestine or for reducing the colonization of the human intestine with pathogenic germs, respectively, preferentially for the purpose of preventing, reducing or curing inflammatory diseases of the human body or preferentially for the purpose of prophylactic or therapeutic treatment of inflammatory diseases of the human body. According to the present aspect, the present invention also relates to the use of a cineole, preferentially 1,8-cineole, for the prophylactic or therapeutic treatment of dysbiosis or of malcolonization of the human intestine with pathogenic germs.

Equally subject matter of the present invention—according to a third aspect of the present invention—is the use of a cineole, preferentially 1,8-cineole, as an intestinal agent.

Yet another object of the present invention—according to a fourth aspect of the present invention—is a method for reducing pathogenic germs in the human intestine or for reducing the colonization of the human intestine with pathogenic germs, respectively, preferentially for the purpose of preventing, reducing or curing inflammatory diseases of the human body or for the purpose of prophylactic or therapeutic treatment of inflammatory diseases of the human body, respectively. According to this aspect, the present invention also relates to a respective method for the prophylactic or therapeutic treatment of dysbiosis or of malcolonization of the human intestine with pathogenic germs. Advantageous further developments and embodiments of this aspect of the invention are provided.

Also subject to the present invention—according to a fifth aspect of the present invention—is further a drug or medicament for (use in) reducing pathogenic germs in the human intestine or for (use in) reducing colonization of the human intestine with pathogenic germs, respectively. According to this aspect, the present invention also relates to a drug or medicament for the prophylactic or therapeutic treatment of dysbiosis or of malcolonization of the human intestine with pathogenic germs. Advantageous further developments and embodiments of this aspect of the invention are provided.

Similarly—according to a sixth aspect of the present invention—it is an object of the present invention to provide a composition for (use in) reducing pathogenic germs in the human intestine or for (use in) reducing colonization of the human intestine with pathogenic germs, respectively. According to this aspect, the present invention also relates to a respective composition for (use in) the prophylactic or therapeutic treatment of dysbiosis or of malcolonization of the human intestine with pathogenic germs, respectively. Advantageous further developments and embodiments of this aspect of the invention are provided.

Finally—according to a seventh aspect of the present invention—the present invention equally relates to a pharmaceutical combination for (use in) reducing pathogenic germs in the human intestine or for (use in) reducing colonization of the human intestine with pathogenic germs, respectively. In this context, the present invention also relates to a pharmaceutical combination for (use in) the prophylactic or therapeutic treatment of dysbiosis or of malcolonization of the human intestine, respectively. Advantageous further developments and embodiments of this aspect of the invention are discussed.

It goes without saying that aspects, embodiments, advantages and the like, which are listed below for the purpose of avoiding repetition with respect to one aspect of the invention, naturally also apply accordingly with respect to the other aspects of the invention, without this requiring separate mention.

Furthermore, with regard to the following description of the present invention, it is also the case that the features of the present invention cited in connection with the particular aspects, embodiments, advantages, examples or the like are also considered disclosed in their combination. Thus, superordinate combinations of individual or several features, which are indicated for respective aspects, embodiments, examples or the like, are also considered disclosed herein.

Furthermore, it goes without saying that in the following disclosure of values, numbers and ranges, the relevant disclosure of values, numbers and ranges are not to be understood as limiting; it goes without saying for the person skilled in the art that, depending on the individual case or application, deviations from the stated range or statements can be made without leaving the scope of the present invention.

In addition, all values or parameters or the like mentioned in the following can basically be determined using standardized or explicitly specified determination methods or using determination methods familiar to those skilled in the art in this field.

Furthermore, it should be noted with regard to all the relative or percentage, especially weight-related, quantitative data mentioned below that, within the framework of the composition according to the invention, these data are to be selected or combined by the person skilled in the art in such a way that in the sum—given the inclusion of further components or ingredients or additives substances or constituents, especially as defined below—always results in 100% or 100 wt.-%. However, this is self-evident for the person skilled in the art.

In addition, the term “drug” or “medicament” (synonymously also “pharmaceutical”), as used in the context of the present invention, is to be understood very comprehensively and includes not only drugs or pharmaceuticals as such (i.e. in terms of drug law), but also, above all, so-called medical devices and, in addition, homeopathics and dietary supplements as well as cosmetics and articles of daily use. In other words, the composition according to the invention may be in the form of a medicament (pharmaceutical), medical product, homeopathic product, nutritional supplement, cosmetic or in the form of an article of daily use.

Having said this, the present invention will now be explained in detail below.

### BRIEF DESCRIPTION OF THE DRAWINGS

FIG. 1 provides a graphical illustration of a principal coordinates analysis (PCoA) of examined stool samples from five patients (1-5) with polyposis nasi, before (without labeling, unfilled circle symbols) and after 2-week ingestion of 1,8-cineole (labeled with “*”, filled circle symbols);

FIG. 2 provides a graphical illustration of the influence of 1,8-cineole on the intestinal microbiome or intestine flora, respectively, of patients with polyposis nasi; with respect to the X-axis, the designation “a” shows the situation or composition of the intestinal microbiome before administration of cineole and the designation “b” shows the situation in this regard after 2 weeks of taking cineole; furthermore, the Y-axis shows the number of sequencing reads performed;

FIG. 3A provides a graphical illustration (based on data n1) showing that the ingestion of 1,8-cineole causes a significant decrease in the cellular complexes of various leukocytes [with “lymphos”=lymphocytes; “monos”=monocytes; “neutros”=neutrophils; “eosinos”=eosinophils in the blood of patients with polyposis nasi; shown in each case is the situation before (“vorher”) and after 2-week administration of 1,8-cineole (“nachher”);

FIG. 3B provides a graphical illustration (based on data n2) showing that the ingestion of 1,8-cineole causes a significant decrease in the cellular complexes of various leukocytes [with “lymphos”=lymphocytes; “monos”=monocytes; “neutros”=neutrophils; “eosinos”=eosinophils in the blood of patients with polyposis nasi; shown in each case is the situation before (“vorher”) and after 2-week administration of 1,8-cineole (“nachher”);

FIG. 4A provides a graphical illustration showing an overall decrease in the population of so-called “classical” monocytes and a clear increase of so-called “intermediate” monocytes in the blood of examined healthy donors (with a=“non-classical” monocytes, b=“intermediate” monocytes and c=“classical” monocytes):

FIG. 4B provides a graphical illustration showing an overall decrease in the population of so-called “classical” monocytes and a clear increase of so-called “intermediate” monocytes in the blood of patients with polyposis nasi (with a=“non-classical” monocytes, b=“intermediate” monocytes and c=“classical” monocytes):

FIG. 4C provides a respective graphical illustration showing an overall decrease in the population of so-called “classical” monocytes and a clear increase of so-called “intermediate” monocytes in the blood of patients situation for patients with polyposis nasi after taking 1,8-cineole for a period of two weeks (with a=“non-classical” monocytes, b=“intermediate” monocytes and c=“classical” monocytes);

FIG. 5A provides a graphical illustration of an increase in monocyte distribution under the influence of 2-week therapy with 1,8-cineole before and after 2-week therapy with 1,8-cineole wherein CD16 neg represents CD16 “classical” monocytes and wherein the respective left circle representation shows the situation before performing the cineole treatment and the respective right box representation shows the situation after performed cineole therapy.

FIG. 5B provides a graphical illustration of a decrease in monocyte distribution under the influence of 2-week therapy with 1,8-cineole before and after 2-week therapy with 1,8-cineole wherein CD16 pos represents CD16 “intermediate” monocytes and wherein the respective left circle representation shows the situation before performing the cineole treatment and the respective right box representation shows the situation after performed cineole therapy.

### DETAILED DESCRIPTION OF THE INVENTION

Thus, the object of the present invention—according to a first aspect of the present invention—is a cineole, preferentially 1,8-cineole, for use in reducing pathogenic germs in the human intestine, especially in the human intestinal flora, and/or for use in reducing the colonization of the human intestine, especially the human intestinal flora, with pathogenic germs, preferentially for the purpose of preventing, reducing or curing inflammatory diseases of the human body and/or preferentially for the purpose of prophylactic and/or therapeutic treatment of inflammatory diseases of the human body.

In the context of the present invention, the term “human intestine” is to be understood very broadly and refers especially to the section of the digestive tract extending from the stomach gate to the anus. Especially, the term in question refers to the small intestine and/or large intestine (colon) section of the human intestine. Thus, the present invention also particularly relates to cineole, preferentially 1,8-cineole, for use in reducing pathogenic germs in the human small intestine and/or colon, especially in the human intestinal flora of the small intestine and/or large intestine, respectively, especially the human intestinal flora of the small and/or large intestine, with pathogenic germs, preferentially for the purpose of preventing, reducing or curing inflammatory diseases of the human body and/or preferentially for the purpose of prophylactic and/or therapeutic treatment of inflammatory diseases of the human body.

In the context of the present invention, the cineole is used or employed especially for reducing the germ load or the germ count of pathogenic germs, respectively, in the human intestine or especially for reducing the germ count of pathogenic germs, relative to the total germ count in the human intestine, especially in the human intestinal flora.

Because—as stated before—the applicant has found in a completely surprising way that cineole, especially 1,8-cineole, is unexpectedly and efficiently suitable as an active ingredient for reducing of pathogenic germs in the human intestine or for the reduction of the colonization of the human intestine with pathogenic germs, respectively, namely especially for the purpose of preventing, reducing or curing inflammatory diseases of the human body or especially for the purpose of prophylactic and/or therapeutic treatment of inflammatory diseases of the human body, respectively. Also in this context the present active ingredient is equally suitable for the prophylactic and/or therapeutic treatment of dysbiosis and/or of related malcolonization of the human intestine.

On the basis of the present invention, a reduction in pathogenic germs in the intestine and a corresponding increase in non-pathogenic or health-promoting germs in the intestine can thus be achieved in a selective manner through the targeted and purposeful use of cineole, preferentially 1,8-cineole, so that on the basis of the conception according to the invention, a positive change or influence on the human intestinal flora is achieved overall with regard to its germ composition. According to the invention, the use of cineole, especially 1,8-cineole, thus results in a significant shift in the microbial and especially the bacterial composition of the intestinal microbiome to the effect that pathogenic and thus harmful germs in the intestinal flora or germs associated with inflammation are sustainably reduced. This also reduces the presence of inflammation-relevant substances or inflammation mediators that originate from the pathogenic germs.

On the basis of the surprisingly found targeted change or influence on the microbiome of the human intestine or the human intestinal flora by cineole, preferentially 1,8-cineole, with the reduction of pathogenic germs, a—without wanting to refer to or limit oneself to this theory—so to speak holistic or systemic effect is achieved in this context as equally found in the context of the present invention in a completely surprising way, namely to the effect that inflammatory diseases or inflammatory processes in the human body as a whole can be positively influenced or controlled in the sense of a reduction or avoidance or cure, namely beyond the human intestine itself as the primary site of action for cineole, especially 1,8-cineole, also in other organs or body regions different from the human intestine, such as for example the mouth/throat region or the throat/nose/ear region (as will be explained below) or on a physical or systemic level as a whole. Due to the positive effect on the intestinal flora according to the invention with the reduction of corresponding pathogenic germs, an improvement of the immune status or the immune system as a whole can be achieved, especially to the extent that inflammatory processes or inflammatory diseases can also be avoided or reduced or cured on this basis.

In this context, the applicant has equally found in a completely surprising manner that inflammatory diseases in the human body, such as for example inflammatory diseases of the mouth/throat area or the ear/throat area, such as rhinosinusitis or the like, can be accompanied by a malcolonization of the human intestine with pathogenic germs or with an excessive presence of pathogenic germs in the human intestinal flora. Consequently, within the scope of the present invention there is also an effective and hitherto undescribed or unthought-of starting point for cineole, preferentially 1,8-cineole, with the targeted influence on the intestinal flora to the effect that, for the first time, inflammatory diseases of the human body associated therewith can also be treated prophylactically or therapeutically.

In this respect, the present invention is particularly concerned with the simultaneous treatment or therapy of abnormal or pathological conditions of the intestinal microbiome or the human intestinal flora, which results in a corresponding reduction or suppression of inflammatory processes in the body, which is further accompanied by a positive effect on inflammatory diseases or conditions of the human body as a whole. This is further accompanied by a positive effect on inflammatory diseases or conditions of the human body as a whole, so that according to the invention, for the first time, a holistic therapy concept is provided with regard to malcolonization of the intestine and related inflammatory processes or diseases of the body.

On the basis of the conception according to the invention with the use of cineole, preferentially 1,8-cineole, for the reduction of pathogenic germs in the human intestine for the purpose of preventing, reducing or curing inflammatory diseases of the human body, systemic inflammatory parameters are also positively influenced especially such that also on this basis an overall positive and especially anti-inflammatory or anti-regulatory effect is present with regard to the treatment of further inflammatory diseases of the human body. This means that, on this basis, there is also an overall positive and especially anti-inflammatory or -regulating effect with regard to the treatment of further inflammatory diseases of the human body, which can be present not only in the area of the intestine itself, but also in further or different parts or regions of the body, as will be explained in detail below.

According to the invention, the targeted use of cineole, preferentially 1,8-cineole, for the purpose of reducing the colonization of the human intestine or the human intestinal flora with pathogenic germs—without wishing to limit or refer to this theory—provides for the first time a holistic or systemic approach with regard to the associated positive effect on inflammatory diseases of the human body, wherein, moreover, according to the invention, a universal and, with regard to the inflammatory disease of the human body, non-specific or generally valid approach is provided, which leads to the possibility of treating a large number of different inflammatory diseases, since, according to the invention, relevant immune or inflammatory regulatory processes can be positively influenced or regulated in a superordinate manner.

In accordance with the invention, the cineole, preferentially 1,8-cineole, has a dysbiotic effect, especially an effect counteracting a malcolonization of the human intestine with pathogenic germs. In this respect, the cineole, preferentially 1,8-cineole, may especially have an effect improving or regulating the systemic immune status of the human body, especially inducing an inhibition of inflammation.

Due to the antidysbiotic effect of cineole, preferentially 1,8-cineole, which was found to be completely surprising in the context of the present invention, an overall anti-inflammatory effect is provided, even at the systemic level, and this effect—without wishing to limit or invoke this theory—has multiple justifications:

Thus, (i) on the one hand, at the primary site of action, namely the human intestine, there is a positive effect on the intestinal epithelium with the immune cells present there, especially on the basis of reducing pathogenic germs in the human intestinal microbiome, so that their negative influence on the intestinal epithelium is reduced or prevented, which leads to an improvement or regulation of the relevant immune status (induction of an active or primary anti-inflammatory mediation). In addition, (ii) on the other hand, the reduction or elimination of pathological germs in the human intestine caused by the administration of cineole, preferentially 1,8-cineole, reduces or prevents the presence or release of corresponding substances relevant to inflammation, such as inflammatory mediators or the like, which originate from the pathogenic germs (induction of passive or secondary anti-inflammatory mediation). Moreover, in the context of the present invention and especially in addition to (i) and (ii), in can also behave in such a way that (iii) excess cineole or 1,8-cineole (i.e. which is not required for the antidysbiotic effect in the intestine or is used up for this purpose, so to speak) can be systemically absorbed or taken up and, for example, develop a corticosteroid-like action profile in the blood system, so that a complementary anti-inflammatory effect can also be exerted on this basis.

The interlocking and reinforcing of the underlying effects thus results in a sustained anti-inflammatory effect, which is not only limited to the intestine itself, but also extends beyond the intestine and thus has a positive effect on the entire body. There is also a regio- or organ-unspecific anti-inflammatory effect or a general systemic anti-inflammatory effect with corresponding downregulation of inflammatory processes overall, so that also on this basis a high efficiency and range of action results, which is relevant for a large number of different inflammatory diseases or inflammatory reactions in the human body.

The special and new as well as inventive application or medical indication for cineole, especially 1,8-cineole, surprisingly discovered by the applicant in the context of the present invention has not been described or recognized in the prior art so far, although cineole, especially 1,8-cineole, is in itself a sufficiently known active ingredient.

The active ingredient cineole used according to the invention, especially 1,8-cineole, is a so-called terpene, especially a monoterpene. Terpenes are generally natural substances which can be isolated from plants or their constituents as components of so-called essential oils in the form of liquids. They are often fragrances and flavorings, which are used in the food or cosmetics industry. However, the use of terpenes for medical purposes is also gaining importance, since pharmacological effects can be demonstrated for a large number of terpenes. Terpenes are formally polymerization products of isoprene, whereby a distinction is made between monoterpenes (C₁₀-units), sesquiterpenes (C₁₅-units), diterpenoids (C₂₀-units), sester terpenes (C₂₅-units), triterpenes (C₃₀-units), tetraterpenes (C₄₀-units) and polyterpenes according to the number of isoprene residues (cf. RÖMPP-Chemielexikon, 10th edition, Georg Thieme Verlag, Stuttgart/New York, 1999, pages 4449 and 4450, keyword “Terpen(oid)e”). In addition, terpenes can also be used as pharmacologically active ingredients, starting materials for the production of drugs or vitamin preparations and in agriculture due to their often bacteriocidal or pesticidal effects. A number of monoterpenes especially have been shown to have pharmacological effects in the treatment of diseases, especially menthol and cineole, especially 1,8-cineole.

Especially, the active ingredient cineole used according to the invention, especially 1,8-cineole, belongs to the bicyclic epoxy monoterpenes, more specifically to the limonene oxides. Synonymous names for 1,8-cineole with the chemical molecular formula C₁₀H₁₈O are eucalyptol, limonene-1,8-oxide, 1,8-epoxy-p-menthane or 1,3,3-trimethyl-2-oxabicyclo[2.2.2]octane. It is a colorless liquid with a spicy, camphor-like odor with a melting point of +1.5° C. and a boiling point of 176 to 177° C., which is insoluble in water but miscible with most organic solvents.

Of course, 1,8-cineole occurs as the main component of eucalyptus oil (eucalyptus oil contains up to 85 wt.-% 1,8-cineole), but it is also found in other plants, such as mint, medicinal sage, thyme, basil and tea tree. In addition, 1,8-cineole is also present, for example, in niaouli, juniperus, piper, cannabis, cajeput, sage oil, myrtle oil and other essential oils.

Technically or pharmaceutically purified 1,8-cineole, which can generally be present with a 99.6% to 99.8% purity, is generally obtained by fractional distillation of eucalyptus oil.

According to the state of the art, 1,8-cineole is applied both topically (e.g. inhalatively) and systemically (e.g. in the form of capsules), usually as a mixed oil together with a variety of other terpenes.

For further details on the active ingredient 1,8-cineole, reference can be made, for example, to RÖMPP Chemielexikon, Georg Thieme Verlag, Stuttgart/New York, 10th edition, Volume 1, 1996, page 752, keyword: “Cineole” and the literature referenced therein.

As a result of the lipophilicity of the cineole, especially 1,8-cineole, preferably applied perorally in accordance with the invention, and especially in an entericcoated (resistant to gastric juice) but small intestine-soluble form, as also indicated below, long-term storage in the relevant epithelial cells of the intestine can also be assumed, so that a long-term and uniform supply of the cineole, especially 1,8-cineole, is also ensured with respect to the intestinal system.

The defined efficacy of the cineole used according to the invention, especially 1,8-cineole, can possibly also be attributed—again without wanting to commit to a specific theory—to the supplementary steroid-like action potential of cineole, especially 1,8-cineole, which was surprisingly found by the applicant, especially with regard to a further or supplementary inhibition of inflammatory mediators, i.e. cineole, especially 1,8-cineole, can also have a steroid-like action potential, especially as a pure substance. Essential mixed oils, on the other hand (which contain cineole in admixture with further terpenes and other active ingredients), stimulate prostaglandin production and show only a reduced inhibition of, for example, leukotriene and cytokine production compared to pure cineole, especially pure 1,8-cineole, as it is preferably used according to the invention; because such mixed oils also contain substances which stimulate cell activity and mediator production and therefore do not have an anti-inflammatory effect, but can cause intolerance reactions, so that essential mixed oils and/or oil mixtures can consequently generally even increase cell activity and induce inflammatory mediator production. In contrast to this, however, cineole, especially 1,8-cineole, especially in pure form, can induce a significant inhibition of mediator production; this can thus also support an anti-inflammatory effect.

In the context of the present invention, the pathogenic germs are especially selected from the group of germs causing (inducing) inflammation, promoting inflammation and being in context with or causing inflammation in the human body, especially from the group of germs producing and/or releasing inflammation-causing and/or inflammation-promoting substances as well as combinations thereof.

Thus, according to the invention, it can be a matter especially of pathogenic germs which, due to their special properties, for example and in a non-limiting manner with regard to the production of inflammation-promoting substances or the like, have a corresponding influence on inflammatory processes in the human body, especially with regard to an inflammation-promoting, evoking or initiating effect. Accordingly, this may also involve inflammatory mediators originating from the pathogenic germs, which especially may also exert systemic or intestine-spanning inflammatory effects (for example, through systemic resorption of these substances or the underlying germs).

According to the invention, it is provided especially that the pathogenic germs are selected from the group of bacteria, especially bacteria negatively affecting health and/or harmful to health, especially from the group of inflammation-causing, inflammation-inducing bacteria and bacteria associated with or causing inflammation in the human body as well as combinations thereof.

According to a preferred embodiment of the invention, the pathogenic germs are selected from the group of bacteria belonging to the family Prevotellaceae, preferentially from the group of bacteria belonging to the genus Prevotella. Bacteria from the Prevotellaceae family or the genus Prevotella are often involved in infections or in inflammatory processes.

Bacteria of the genus Prevotella are especially gram-negative obligate anaerobic species, which are pleomorphic rod-shaped, immobile and sporeless and which exhibit a chemoorganotrophic fermentation metabolism, the main end products of which are acetate and succinate. The genus Prevotella generally includes about forty different species, which can often cause or aggravate various inflammatory processes, for example of the oral cavity or, for example, of the respiratory tract.

The applicant has surprisingly found out that bacteria of the family Prevotellaceae, especially of the bacterial genus Prevotella, are present to an increased extent in the intestine of patients suffering from polyposis nasi, wherein the proportion or the number of these bacteria can be sustainably reduced by administration of cineole, preferentially 1,8-cineole, in an equally completely surprising manner, as will be shown below.

In the context of the present invention, it may also be particularly the case that cineole, preferentially 1,8-cineole, is further or additionally or equally used or employed to increase, multiply or support health-promoting or non-pathogenic, preferably health-promoting, germs in the human intestine, especially in the human intestinal flora. The applicant has found, both unexpectedly and surprisingly, that the proportion or number of such positive germs in the human intestine can be significantly increased under cineole or 1,8-cineole application.

In this respect, in addition to a reduction of pathogenic germs, an increase or propagation or support of health-promoting or non-pathogenic germs can thus also be achieved by the targeted administration of cineole, preferentially 1,8-cineole, so that also on this basis the composition of the microbiome of the human intestine or the intestinal flora can be improved or normalized overall. Thus, in the context of the present invention, the cineole, preferentially 1,8-cineole, used also has a multiple effect, namely on the one hand with regard to a reduction of pathogenic germs and a targeted support of health-promoting or non-pathogenic germs in the human intestine.

In this context, it can thus be provided according to the invention that the cineole, preferentially 1,8-cineole, is further or additionally or equally used or employed for increasing the germ load or the germ number or the germ proportion, relative to the total germ number in the human intestine, especially in the human intestinal flora, of health-promoting or non-pathogenic, preferably health-promoting, germs in the human intestine, especially in the human intestinal flora.

Especially, the cineole, preferentially 1,8-cineole, may further and/or additionally and/or equally be used or employed for use in increasing the colonization of the human intestine, especially the human intestinal flora, with health-promoting or non-pathogenic, preferably health-promoting, germs.

In general, in the context of the present invention, the health-promoting or non-pathogenic, preferably health-promoting, germs may be selected from the group of inflammation-reducing, anti-inflammatory and inflammation-inhibiting germs, especially from the group of germs producing and/or releasing anti-inflammatory and inflammation-inhibiting substances as well as combinations thereof. Especially, the health-promoting or non-pathogenic germs in question may be those germs which produce or release anti-inflammatory substances. This can also lead to a targeted reduction or inhibition of inflammatory processes in the human body.

By reducing pathogenic germs, especially with a proinflammatory effect, on the one hand, and supporting health-promoting or non-pathogenic germs, especially with an anti-inflammatory effect, on the other hand, a dual approach is thus achieved within the scope of the present invention with regard to an inflammation-reducing or repressing or inhibiting effect in connection with the administration of cineole, preferentially 1,8-cineole, so that also from this point of view there is a very high efficacy, not only with regard to inflammatory processes or diseases in the human intestine itself, but also with regard to systemic or local inflammations which affect other parts of the human body or parts of the human body different from the human intestine, as will be further explained below.

In accordance with a preferred embodiment according to the invention, the health-promoting or non-pathogenic, preferably health-promoting, germs are selected from the group of bacteria, especially health-positive and/or health-promoting bacteria, preferentially from the group of inflammation-reducing, anti-inflammatory and inflammation-inhibiting bacteria as well as combinations thereof.

Especially, it may be provided in the context of the present invention that the health-promoting or non-pathogenic, preferably health-promoting, germs are selected from the group of bacteria of the family Ruminococcacea, preferentially from the group of bacteria of the genus Ruminococcus. As far as bacteria of the Ruminococcacea family or the genus Ruminococcus are concerned, these are especially those bacteria which produce anti-inflammatory short-chain fatty acids, such as butyrate, so that the bacteria in question can be said to have a positive effect with regard to reducing inflammatory processes in the human body.

According to the invention, it may also be provided that the health-promoting or non-pathogenic, preferably health-promoting, germs are selected from the group of bacteria of the families Bacteroidaceae, Lachnospiraceae and Bifidobacteriaceae, especially Bacteroidaceae and Lachnospiraceae, preferentially Bacteroidaceae as well as combinations thereof. Especially, according to the invention, it may be specifically provided that the health-promoting or non-pathogenic, preferably health-promoting, germs are selected from the group of bacteria of the genus Bacteroides, Lachnospira and Bifidobacterium, especially Bacteroides and Lachnospira, preferentially Bacteroides as well as combinations thereof. Bacterial species belonging to the aforementioned families or genera can also be associated with a positive effect with respect to human intestinal flora or with anti-inflammatory effects.

With regard to the cineole, preferentially 1,8-cineole, it can also be provided in accordance with the invention that it is further and/or additionally and/or equally used or employed for the regeneration and/or rehabilitation of the microbial colonization of the human intestine, especially of the human intestinal flora. This is accompanied especially by a reduction in undesirable or pathogenic germs or by an increase in desirable or non-pathogenic, especially health-promoting, germs in the human intestine or in the human intestinal flora.

According to the invention, the cineole, preferentially 1,8-cineole, can also be used or employed for increasing the germ diversity or for diversifying the germ colonization, respectively, of the human intestine, especially of the human intestinal flora. In this context, the increase in germ diversity or diversification is accompanied, especially, by an increase in the number of species of the underlying germs or bacteria, especially health-promoting or non-pathogenic, preferably health-promoting, germs or bacteria.

According to the invention, the cineole, preferentially 1,8-cineole, can be used or employed especially for reducing the germ load and/or the germ count of pathogenic germs in the human intestine and/or for reducing the germ count of pathogenic germs, relative to the total germ count in the human intestine, especially in the human intestinal flora.

In this context, the cineole, preferentially 1,8-cineole, can especially also be used or employed for increasing the germ load or the germ number or the germ proportion, relative to the total germ number in the human intestine, of health-promoting or non-pathogenic, preferably health-promoting, germs, especially as defined above, in the human intestine, especially in the human intestinal flora.

As far as the cineole, preferentially 1,8-cineole, is further concerned with regard to its use according to the invention, the cineole, preferentially 1,8-cineole, can further and/or additionally and/or equally be used or employed as an anti-inflammatory agent, especially as a systemically acting anti-inflammatory agent.

Especially, the cineole, preferentially 1,8-cineole, can further or additionally or equally be used or employed, respectively, as an active ingredient having an anti-inflammatory effect on the especially peripheral blood, preferentially having an anti-inflammatory effect on immune cells of the especially peripheral blood.

With regard to the anti-inflammatory effect in this respect, according to the invention—without wishing to limit or refer to this theory—it may especially be a matter of a certain indirect effect of cineole, preferentially 1,8-cineole, namely in so far as a reduction in the germ load of pathogenic germs and/or an increase in colonization with health-promoting or non-pathogenic germs, preferably health-promoting, germs of the human intestine, especially the intestinal flora, which may be accompanied by an improvement in the condition of the intestinal epithelium, which is accompanied by corresponding anti-inflammatory effects also at the systemic level. Moreover, as stated before, it can be basically the case that excess cineole, which is not consumed for the antidysbiotic effect, is absorbed and causes a complementary systemic effect.

According to the invention, the cineole, preferentially 1,8-cineole, may further or additionally or equally be used or employed for suppression or attenuation of inflammation mediation (i.e. inflammation mediation or inflammation cascade) occurring in the context of inflammatory diseases of the human body.

Especially, the cineole, preferentially 1,8-cineole, may exert a suppressive or attenuating effect towards an inflammatory mediation occurring in the context of the inflammatory diseases of the human body.

Due to the, so to speak, universal or organ-spanning effect of cineole, preferentially 1,8-cineole, especially on the basis of the reduction of pathogenic germs or the increase of health-promoting or non-pathogenic germs in the human intestine, especially in the human intestinal flora, with the systemic effects in this respect, a large number of different inflammatory diseases can be reduced, prevented or cured within the scope of the present invention, since, on the basis of the underlying mechanisms, there is, so to speak, a universal and, with regard to the specific underlying inflammatory disease, independent and thus generally valid principle of the inhibition of inflammation, namely especially in connection with or as a result of the targeted influence of the human intestinal system on the basis of the germs or bacteria present in the human intestine or in the human intestinal flora.

According to the invention, also against this background, it can behave especially in such a way that the inflammatory diseases of the human body are associated with the pathogenic germs in the human intestine, especially in the human intestinal flora or are caused and/or induced and/or enhanced thereby.

Especially, according to the invention, it can behave in such a way that the inflammatory diseases of the human body are associated with an excessive and/or abnormal and/or pathological malcolonization, especially overcolonization, of the human intestine, especially of the human intestinal flora, with the pathogenic germs or are caused and/or induced and/or enhanced thereby.

Especially, the diseases can also be inflammatory diseases which are associated with a dysbiosis and/or a malcolonization of the human intestine, especially of the human intestinal flora, with pathogenic germs or are caused and/or induced and/or intensified thereby.

According to the invention, it may be generally such that the inflammatory diseases of the human body are local inflammatory diseases or systemic inflammatory diseases.

According to the invention, it is also possible especially for the inflammatory diseases of the human body to be acute inflammatory diseases or chronic inflammatory diseases. In a preferred manner, the inflammatory diseases are chronic inflammatory diseases.

As previously noted, the underlying diseases may be selected from a variety of diseases. Especially, the inflammatory diseases of the human body may be selected from the group of (i) inflammatory diseases of the upper respiratory tract, especially inflammatory diseases of the nose and/or the paranasal sinuses and/or the mouth/throat; (ii) inflammatory diseases of the throat/nose/ear region; (iii) inflammatory diseases of the lower respiratory tract, especially inflammatory pulmonary diseases (lung diseases), inflammatory bronchial diseases and inflammatory bronchopulmonary diseases; (iv) inflammatory diseases of the gastrointestinal tract, especially of the intestine; (v) inflammatory diseases of the skin; (vi) inflammatory diseases of the genitourinary tract, especially inflammatory diseases of the urinary tract; (vii) inflammatory rheumatoid diseases, especially rheumatism and rheumatoid arthritis, and rheumatic diseases; and (viii) inflammatory diseases of (further or other) as well as combinations thereof.

Especially, the inflammatory diseases of the human body may be selected from the group of rhinitis, sinusitis, rhinosinusitis and nasal polyps (polyposis nasi etsinuum) as well as combinations thereof.

Especially, the inflammatory diseases of the human body may be selected from the group of inflammatory diseases of the ear, preferably otitis, preferably otitis media as well as combinations thereof.

Especially, with regard to rhinosinusitis, it can be, for example, rhinosinusitis with nasal polyps (polyposis nasi etsinuum) (RSwNP), especially chronic rhinosinusitis with nasal polyps (polyposis nasi etsinuum) (CRSwNP). In principle, however, according to the invention, it can also be rhinosinusitis without nasal polyps (RSsNP) or chronic rhinosinusitis without nasal polyps (CRSsNP).

According to the invention, the inflammatory diseases of the human body may also be selected from the group of inflammatory diseases of the ear, preferentially otitis, preferably otitis media as well as combinations thereof. Especially, this may involve polypous otitis media, especially chronic polypous otitis media. Especially, this may also be a chronic polypous otitis media with cholesteatoma or without cholesteatoma.

Furthermore, the inflammatory diseases of the human body can be selected from the group of bronchitis, bronchial asthma and chronic obstructive pulmonary disease (COPD) as well as combinations thereof.

Especially, the inflammatory diseases of the human body may be selected from the group of Crohn's disease, ulcerative colitis and inflammatory bowel diseases such as Inflammatory Bowel Disease (IBD) as well as combinations thereof.

According to the invention, it may further be provided that the inflammatory diseases of the human body are selected from the group consisting of eczema and dermatitis as well as combinations thereof.

The inflammatory diseases of the human body may also be selected from the group of glomerulonephritis, pyelonephritis, cystitis and iritis as well as combinations thereof.

Similarly, according to the invention, it may be the case that the inflammatory diseases of the human body are selected from the group of inflammatory diseases of the biliary tract, especially cholecystitis and cholangitis as well as combinations thereof.

According to a particular embodiment of the present invention according to this aspect of the invention, it is especially provided that the cineole, preferentially 1,8-cineole, is applied systemically, especially perorally or parenterally, preferentially perorally. In this context, it may especially be provided that the cineole, preferentially 1,8-cineole, is prepared for systemic, especially peroral or parenteral, preferentially peroral, application. In this way, high active doses or active ingredient levels (i.e. active ingredient levels or concentrations) especially in the region of the intestine are achieved, so that particularly good efficacy or efficiency can be realized.

Especially, the cineole, preferentially 1,8-cineole, may be applied in the form of a dosage form to be administered perorally and/or wherein the cineole, preferentially 1,8-cineole, is prepared in a dosage form to be administered perorally.

According to a special embodiment of the present invention according to this aspect, it may especially also be provided in this context that the cineole, preferentially 1,8-cineole, is applied as an entericcoated (resistant to gastric juice) but small intestine soluble systemic dosage form, preferably as a capsule, dragee, pill, tablet or the like. Especially, the cineole, preferentially 1,8-cineole, may be prepared for administration as an entericcoated (resistant to gastric juice) but small intestine soluble systemic dosage form, preferably as a capsule, dragee, pill, tablet or the like. Due to the entericcoated (resistant to gastric juice) but small intestine soluble systemic dosage form, respectively, the active dose or the active ingredient level in the region of the intestine can be further increased due to the targeted release only “on site”. In general, it is thus provided in the context of the present invention that the peroral dosage form, especially in the form of a capsule, is designed to be entericcoated (resistant to gastric juice) but soluble in the small intestine. This achieves a particularly optimal release profile since the active ingredient is released in a targeted and purposeful manner only in the intestine.

The definitions of the terms “entericcoated (resistant to gastric juice)” and “small intestine soluble” used in the context of the present invention and the related test methods are given in European Pharmacopoeia 7.0, April 2010: 1502, pages 707 to 709, keyword “Capsules”, subchapter “Gastro-Resistant Capsules” and European Pharmacopoeia 7.1, April 2011: 20901, pages 3331 and 3332, keyword “2.9.1 Disintegration of Tablets and Capsules”.

In the context of the present invention, the term “entericcoated (resistant to gastric juice)” is to be understood especially as meaning that the capsules can be kept, especially stirred, for at least two hours in 0.1 N hydrochloric acid, which is heated to temperatures of 35 to 39° C., with constant mixing, without the capsules showing signs of decomposition or cracking or other damage.

On the other hand, in the context of the present invention, the term “small intestine soluble” means especially that the capsules are decomposed in an aqueous phosphate buffer solution, which is adjusted to a pH of about 6.8, with stirring at temperatures in the range of 35 to 39° C. within one hour to such an extent that the active ingredient is released.

For the application of cineole, especially 1,8-cineole, various preparations are commercially available, especially on the basis of generally entericcoated (resistant to gastric juice), but small intestine soluble dosage forms or capsules (e.g. Soledum® capsules or Soledum® forte capsules, distributed by Cassella-med GmbH & Co. KG and Maria Clementine Martin Klosterfrau Vertriebsgesellschaft mbH, respectively, Cologne, Germany).

According to the invention, it may be provided that the cineole, preferentially 1,8-cineole, is administered in pharmaceutically effective or therapeutically effective amounts, respectively and/or wherein the cineole, preferentially 1,8-cineole, is adapted for administration in pharmaceutically effective or therapeutically effective amounts, respectively.

In the context of the present invention, it is particularly intended that the cineole, preferentially 1,8-cineole, is administered at a daily dose in the range of from 1 to 5,000 mg/diem, especially in the range of from 2 to 3,000 mg/diem, preferentially in the range of from 5 to 2,500 mg/diem, preferably in the range of from 10 to 2,000 mg/diem, more preferably in the range of from 50 to 1,500 mg/diem, and/or wherein the cineole, preferentially 1,8-cineole, is prepared for administration at a daily dose in the range of from 1 to 5,000 mg/diem, especially in the range of from 2 to 3,000 mg/diem, preferentially in the range of from 5 to 2,500 mg/diem, preferably in the range of from 10 to 2,000 mg/diem, more preferably in the range of from 50 to 1,500 mg/diem.

Especially, it may also be provided in the context of the present invention according to this aspect of the invention according to a particular embodiment that the cineole, preferentially 1,8-cineole, is present and/or administered as a pure substance. In this context, the cineole, preferentially 1,8-cineole, may be present and/or administered with a purity of at least 95 wt.-%, especially at least 96 wt.-%, preferably at least 97 wt.-%, more preferably at least 98 wt.-%, even more preferably at least 99 wt.-%, most preferably at least 99.5 wt.-%, based on the cineole, preferentially 1,8-cineole. Especially, it may also be provided in this context that the cineole, preferentially 1,8-cineole, is free of other terpenes or that the cineole, preferentially 1,8-cineole, does not contain any other terpenes.

In other words, in the context of the present invention according to this aspect of the invention, according to a particular embodiment, it may especially be provided that the cineole, preferentially 1,8-cineole, is present and/or administered in the absence of any other (further) terpenes.

According to a particular embodiment according to the invention, it may thus be provided especially that the cineole, preferentially 1,8-cineole, is present and/or administered as a pure substance and/or in the absence of any other (further) terpenes.

As described above, it can therefore be preferred in accordance with the invention that the monoterpene-based active ingredient (i.e. cineole, preferentially 1,8-cineole) is used in the form of a pure substance, i.e. as the sole or technically isolated/purified active ingredient or free from any other terpenes. In this way, for example, an always constant dosage or an always constant content of active ingredient can be ensured in the dosage form provided according to the invention. Especially, high active ingredient doses or levels can also be realized at the site of action in this way. As a result of the preferred use according to the invention of cineole, especially 1,8-cineole, in pure form or free of other terpenes, a particularly defined active profile is also ensured with regard to the underlying indication. As previously stated, administration in pure form also provides a defined steroid-like efficacy profile.

According to yet another embodiment of the present invention, it may be provided that the cineole, preferentially 1,8-cineole, is present or administered together with at least one physiologically acceptable carrier (excipient).

According to yet another embodiment of the present invention, it may be provided that the cineole, preferentially 1,8-cineole, is preferably present or administered as a pure substance and/or in the absence of any other (further) terpenes, together with at least one physiologically acceptable carrier (excipient).

Especially, it may be provided that the physiologically acceptable carrier (excipient) is miscible with and/or soluble in cineole, preferably 1,8-cineole.

Especially, the physiologically acceptable carrier (excipient) may be present in liquid or solid, preferentially liquid, aggregate state at 20° C. and at atmospheric pressure.

In this context, it may be further provided that the physiologically acceptable carrier (excipient) is selected from the group of fatty acid esters, preferably triglycerides of fatty acids, more preferably medium chain triglycerides (MCT), even more preferably triglycerides of C₆-C₁₂-fatty acids.

Especially, medium-chain triglycerides, such as are preferably used according to the invention as a carrier or excipient for the monoterpene-containing active ingredient, are especially semisynthetic neutral glycerol esters of saturated, generally unbranched monocarboxylic acids of medium chain length (i.e. C₆-C₁₂-chains). Especially, the term medium-chain triglycerides refers to mixtures of triglycerides of saturated fatty acids, mainly caprylic acid (octanoic acid) and capric acid (decanoic acid). Medium-chain triglycerides can generally be produced from oil extracted from the solid and dried part of the endosperm of Cocos nucifera L. and/or from the dried endosperm of Elaeis guineenses Jacq. For further details on the concept of medium-chain triglycerides, reference can be made, for example, to the Ph. Eur. monograph, 6th edition, basic work 2008, pages 4224 to 4226, as well as to the Zeitschrift für Ernahrungswissenschaft, volume 13, issue 1/2, 1973, pages 6 ff, D. Sailer et al. “Mittelkettige Triglyceride—Klinische Physiologie und Anwendung”).

According to the invention, it is preferred if the carrier or excipient is used in an active ingredient/carrier quantitative ratio in the range from 1,000:1 to 1:1,000, especially 100:1 to 1:100, preferentially 50:1 to 1:50, more preferably 10:1 to 1:10, even more preferably 5:1 to 1:2, most preferably 3:1 to 1:1. By mixing the active ingredient (i.e. cineole, especially 1,8-cineole) with the excipient, a significantly improved compatibility of the active ingredient with the other ingredients of the dosage form and an improved stability, especially storage stability, are achieved.

According to an equally further particular embodiment of the present invention, it may be provided that the cineole, preferentially 1,8-cineole, is present and/or administered in the form of a composition, especially a pharmaceutical composition, especially together with at least one pharmaceutically acceptable and/or physiologically harmless excipient (carrier).

In this context, it may be particularly envisaged that the composition contains the cineole, preferentially 1,8-cineole, as the sole active ingredient, especially as the sole pharmaceutical active ingredient.

Furthermore, it may be provided in this context especially that the composition contains the cineole, preferentially 1,8-cineole, as a pure substance, preferentially with a purity of at least 95 wt.-%, especially at least 96 wt.-%, preferably at least 97 wt.-%, more preferably at least 98 wt.-%, even more preferably at least 99 wt.-%, most preferably at least 99.5 wt.-%, based on the cineole, preferentially 1,8-cineole, and/or preferably free of any other terpenes.

Equally, it may be provided thereby that the composition contains the cineole, preferentially 1,8-cineole, at least substantially free of any other terpenes and/or especially wherein the composition contains at least substantially no other terpene and/or especially wherein the composition is at least substantially free of terpenes other than cineole, preferentially 1,8-cineole.

Furthermore, it may be provided in this context that the composition contains the cineole, preferentially 1,8-cineole, in effective, especially pharmaceutically and/or therapeutically effective, amounts.

Likewise, in this context, it may be provided according to the invention that the composition contains the cineole, preferentially 1,8-cineole, in relative amounts, based on the composition, in the range of from 0.0001 to 80 wt.-%, especially in the range of from 0.001 to 75 wt.-%, preferentially in the range of from 0.005 to 70 wt.-%, preferably in the range of from 0.01 to 60 wt.-%, more preferably in the range of from 0.05 to 55 wt.-%, even more preferably in the range of from 0.1 to 50 wt.-%.

Finally, in accordance with the invention, it may also be provided in this context that the at least one pharmaceutically acceptable and/or physiologically harmless excipient (carrier) is miscible with cineole, preferentially 1,8-cineole, and/or soluble therein, preferentially wherein the at least one pharmaceutically acceptable and/or physiologically harmless excipient (carrier) is in liquid or solid, preferentially liquid, aggregate state at 20° C. and at atmospheric pressure and/or preferentially wherein the at least one pharmaceutically acceptable and/or physiologically harmless excipient (carrier) is selected from the group of fatty acid esters, preferably triglycerides of fatty acids, more preferably medium-chain triglycerides (MCT), even more preferably triglycerides of C₆-C₁₂-fatty acids.

According to a particular embodiment of the present invention, the cineole, preferentially 1,8-cineole, may be present and/or administered in liposome-encapsulated and/or liposome-packaged form.

According to another particular embodiment of the present invention, the cineole, preferentially 1,8-cineole, may be present and/or administered in micellar form and/or in a micellar dosage form.

According to the invention, it may further be provided that the cineole, preferentially 1,8-cineole, is present and/or administered as a single agent and/or as a monopreparation.

According to the invention, it may be provided in this context that the cineole, preferentially 1,8-cineole, is present or administered without or in the absence of further active ingredients other than cineole, preferentially 1,8-cineole, respectively.

In contrast, however, it can also be provided according to the invention that the cineole, preferentially 1,8-cineole, is present and/or administered together with at least one further active ingredient and/or as a co-therapeutic agent.

In this context, it may be provided especially that the further active ingredient is selected from (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms positively influencing the intestinal flora; as well as combinations thereof.

According to a particularly preferred embodiment, it can be provided especially that the further active ingredient is used and/or administered separately, especially spatially separated, from the cineole, preferentially 1,8-cineole, but functionally coherent therewith, especially in the form of a kit (kit-of-part).

In the aforementioned manner (i.e. combination or co-administration with at least one further active ingredient), especially the effect or efficiency of the further active ingredient can be increased. At the same time, the interaction can also significantly increase the sensitivity to the further active ingredient, as is the case, for example, for active ingredients that inhibit inflammation. Similarly, the efficacy can be improved, for example, by combined administration with prebiotics or microorganisms that positively influence the intestinal flora, since this can further influence or build up the intestinal flora with regard to the preference of non-pathogenic or health-promoting germs or with regard to a further targeted reduction of pathogenic germs (as may also be the case, for example, for the use of preferably special antibiotics).

Especially, the cineole, preferentially 1,8-cineole, may be present and/or administered together with at least one prebiotic. In this context, the prebiotic may be a substance which especially specifically promotes the growth of microorganisms positively influencing the intestinal flora. In this context, it may also behave in such a way that the prebiotic is selected from the group of especially natural polysaccharides, especially natural oligosaccharides, and sugar alcohols.

Especially, the prebiotic may be gum arabic.

Similarly, the prebiotic in this context may be selected from the group of fructooligosaccharides and galactooligosaccharides, especially each in the form of tri- to pentasaccharides as well as combinations thereof. Especially, the prebiotic may be selected from the group of inulin, sucrose, stachyose, raffinose, lactulose as well as combinations thereof.

Furthermore, it may be provided in the context of the present invention that the cineole, preferentially 1,8-cineole, is present and/or administered together with microorganisms positively influencing the intestinal flora. Especially, these may be health-promoting or non-pathogenic microorganisms as such. For example, the microorganisms may be selected from the group of yeasts and bacteria. In this context, it can be provided according to the invention especially that the microorganisms are selected from the group of species of the genus Saccharomyces, especially Saccharomyces boulardii and Saccharomyces cervesiae, preferentially Saccharomyces boulardii as well as combinations thereof. Similarly, in accordance with the invention, it can be provided in this context that the microorganisms are selected from the group of species of the genus Bifidobacterium, Lactobacillus, Enterococcus, Escherichia, Streptococcus, especially each in the form of their species positively influencing the intestinal flora as well as combinations thereof.

In general, the microorganisms can be present and/or administered as a dry substance, especially biologically active dry substance, especially in lyophilized form and/or as lyophilizate. This is particularly conducive to handling while maintaining high efficacy.

The present invention further relates according to the present aspect also to a cineole, preferentially 1,8-cineole, especially cineole for use as previously defined, for use in the prophylactic and/or therapeutic treatment of dysbiosis and/or of malcolonization of the human intestine, especially of the human intestinal flora, with pathogenic germs.

Especially, regeneration and/or sanitation of the microbiome of the human intestine, especially of the human intestinal flora, can be carried out in the context of the treatment of dysbiosis or of malcolonization of the human intestine, especially of the human intestinal flora, with pathogenic germs.

In this respect, too, an optimization or regeneration of the intestinal flora can be achieved within the scope of the present invention and especially also against the background of or for the purpose of preventing, reducing or curing inflammatory diseases of the human body or especially for the purpose of prophylactic and/or therapeutic treatment of inflammatory diseases of the human body. Also in this context, cineole, especially 1,8-cineole, has an antidysbiotic effect. Consequently, cineole, especially 1,8-cineole, can also act as an antidysbiotic in this respect.

The present invention, both according to the first aspect of the present invention and according to all other aspects of the present invention, is thus associated with a large number of advantages and special features which make the therapy concept according to the invention unique and special, especially highly efficient, and this at the same time with good tolerability and simple applicability of the active ingredient present or used according to the invention.

For further details on this aspect of the invention, reference may also be made to the following explanations of the other aspects of the invention, which explanations apply equally to the present aspect of the invention.

A further object of the present invention—according to a second aspect of the present invention—is furthermore the inventive use of a cineole, preferentially 1,8-cineole, for reducing pathogenic germs in the human intestine, especially in the human intestinal flora, and/or for reducing the colonization of the human intestine, especially the human intestinal flora, with pathogenic germs, preferentially for the purpose of preventing, reducing or curing inflammatory diseases of the human body and/or preferentially for the purpose of prophylactic and/or therapeutic treatment of inflammatory diseases of the human body.

In the context of the use according to the present aspect of the invention, it is particularly intended that the active ingredient or the cineole, preferentially 1,8-cineole, is applied perorally, especially in the form of an entericcoated (resistant to gastric juice) but small intestine-soluble dosage form or composition.

In accordance with the present aspect, the present invention further relates to the use of a cineole, especially 1,8-cineole, for producing a drug or medicament for reducing pathogenic germs in the human intestine, especially in the human intestinal flora, and/or for producing a drug or medicament for reducing the colonization of the human intestine, especially the human intestinal flora, with pathogenic germs, preferentially for the purpose of preventing, reducing or curing inflammatory diseases of the human body and/or preferentially for the purpose of prophylactic and/or therapeutic treatment of inflammatory diseases of the human body.

Similarly, according to the present aspect, the present invention also relates to the use of a cineole, especially 1,8-cineole, especially the use as defined hereinabove, (for producing a drug) for the prophylactic and/or therapeutic treatment of dysbiosis and/or of malcolonization of the human intestine, especially of the human intestinal flora, with pathogenic germs.

In this regard, the cineole, preferentially 1,8-cineole, particularly the drug or medicament can be administered in pharmaceutically effective or therapeutically effective amounts.

Especially, the cineole, preferentially 1,8-cineole, especially the drug or medicament may be prepared for administration in pharmaceutically effective or therapeutically effective amounts.

In addition, the cineole, preferentially 1,8-cineole, especially the drug or medicament, can be applied systemically.

According to the invention, in this context the cineole, preferentially 1,8-cineole, especially the drug or medicament or the composition can be used or applied together with at least one further active ingredient, especially wherein the further active ingredient is selected from the group of (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms positively influencing the intestinal flora; as well as combinations thereof.

For further details on this aspect of the invention, reference can be made to the explanations on the further aspects according to the invention, wherein these explanations apply equally to the present aspect of the invention.

Similarly, it is an object of the present invention—according to a third aspect of the present invention—to the inventive use of a cineole, especially 1,8-cineole, as an intestinal agent for reducing pathogenic germs in the human intestine, especially in the human intestinal flora; and/or for use in reducing colonization of the human intestine, especially the human intestinal flora, with pathogenic germs, preferentially for the purpose of preventing, reducing or curing inflammatory diseases of the human body and/or preferably for the purpose of prophylactic and/or therapeutic treatment of inflammatory diseases of the human body; and/or for the prophylactic and/or therapeutic treatment of dysbiosis and/or of especially bacterial overgrowth of the human intestine, especially of the human intestinal flora.

In this regard, the cineole, preferentially 1,8-cineole, may be administered in pharmaceutically effective or therapeutically effective amounts.

Especially, the cineole, preferentially 1,8-cineole, may be prepared for administration in pharmaceutically effective or therapeutically effective amounts.

In addition, the cineole, preferentially 1,8-cineole, can be applied systemically.

According to the invention, in this context the cineole, preferentially 1,8-cineole, or the composition, can be used or applied together with at least one further active ingredient, especially wherein the further active ingredient is selected from the group of (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms positively influencing the intestinal flora; as well as combinations thereof.

For further details on this aspect of the invention, reference can be made to the explanations on the further aspects according to the invention, whereby these explanations apply equally to the present aspect of the invention.

Also an object of the present invention—according to a fourth aspect of the present invention—is the inventive method for reducing pathogenic germs in the human intestine, especially in the human intestinal flora, and/or for reducing the colonization of the human intestine, especially of the human intestinal flora, with pathogenic germs, preferentially for the purpose of preventing, reducing or curing inflammatory diseases of the human body and/or preferentially for the purpose of prophylactic and/or therapeutic treatment of inflammatory diseases of the human body, wherein in the method an amount of cineole, preferentially 1,8-cineole, reducing and/or lowering the germ load and/or the germ number of the pathogenic germs in the intestine is administered to a human patient especially comprising a malcolonization, especially an overcolonization, of the intestine, especially of the intestinal flora, with the pathogenic germs; and/or wherein in the method an amount of cineole, preferentially 1,8-cineole, causing a reduction of the germ proportion of the pathogenic germs relative to the total germ number in the intestine, especially in the intestinal flora is administered to a human patient comprising especially a malcolonization, especially an overcolonization, of the intestine, especially of the intestinal flora, with the pathogenic germs; and/or wherein in the method a pharmaceutically effective and/or therapeutically effective amount of cineole, preferentially 1,8-cineole, is administered to a human patient especially comprising a malcolonization, especially an overcolonization, of the intestine, especially of the intestinal flora, with the pathogenic germs.

In this regard, the cineole, preferentially 1,8-cineole, may be administered in pharmaceutically effective or therapeutically effective amounts.

Especially, the cineole, preferentially 1,8-cineole, may be prepared for administration in pharmaceutically effective or therapeutically effective amounts.

In addition, the cineole, preferentially 1,8-cineole, can be applied systemically.

According to the invention, in this context the cineole, preferentially 1,8-cineole, or the composition, can be used or applied together with at least one further active ingredient, especially wherein the further active ingredient is selected from the group of (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms positively influencing the intestinal flora; as well as combinations thereof.

According to this aspect, the present invention also relates to the inventive method, especially to the method defined above, for the prophylactic and/or therapeutic treatment of dysbiosis and/or of malcolonization of the human intestine, especially of the human intestinal flora, with pathogenic germs, wherein in the method a pharmaceutically effective and/or therapeutically effective amount of cineole, preferentially 1,8-cineole, is administered to a patient exhibiting the dysbiosis and/or the malcolonization and/or suffering therefrom, especially to a patient comprising the malcolonization, especially overgrowth, of the intestine, especially of the intestinal flora, with the pathogenic germs and/or to a patient suffering from dysbiosis.

For further details on this aspect of the invention, reference can be made to the explanations on the other aspects of the invention, whereby these explanations apply equally to the present aspect of the invention.

Also subject to the present invention—according to a fifth aspect of the present invention—is further the drug or medicament, especially intestinal treatment agent, for (use in) reducing pathogenic germs in the human intestine, especially in the human intestinal flora, and/or for (use in) reducing the colonization of the human intestine, especially the human intestinal flora, with pathogenic germs, preferentially for the purpose of preventing, reducing or curing inflammatory diseases of the human body and/or preferentially for the purpose of prophylactic and/or therapeutic treatment of inflammatory diseases of the human body, wherein the drug or medicament, especially intestinal treatment agent, contains cineole, preferentially 1,8-cineole, together with at least one pharmaceutically acceptable and/or physiologically harmless excipient (carrier).

According to this aspect, the present invention relates to the inventive drug or medicament, especially intestinal treatment agent, especially as defined hereinabove, for the prophylactic and/or therapeutic treatment of dysbiosis and/or of malcolonization of the human intestine, especially of the human intestinal flora, with pathogenic germs, wherein the drug or medicament, especially intestinal treatment agent, contains cineole, preferentially 1,8-cineole, together with at least one pharmaceutically acceptable and/or physiologically harmless excipient (carrier).

In this respect, it may be provided according to the invention that the drug or medicament contains the cineole, preferentially 1,8-cineole, as the sole active ingredient, especially as the sole pharmaceutical active ingredient.

According to the invention, it may also be provided that the drug or medicament contains the cineole, preferentially 1,8-cineole, as a pure substance, preferentially with a purity of at least 95 wt.-%, especially at least 96 wt.-%, preferably at least 97 wt.-%, more preferably at least 98 wt.-%, even more preferably at least 99 wt.-%, most preferably at least 99.5 wt.-%, based on the cineole, preferentially 1,8-cineole, and/or preferentially free of any other terpenes.

Especially, the drug or medicament may contain the cineole, preferentially 1,8-cineole, free of any other terpenes. Especially, the drug or medicament may contain no other terpene. Especially, the drug or medicament may be free of terpenes other than cineole, preferentially 1,8-cineole.

According to the invention, the drug or medicament may contain the cineole, preferentially 1,8-cineole, in effective, especially pharmaceutically and/or therapeutically effective, amounts.

Especially, it may be provided in this context that the medicament or drug according to the invention contains the cineole, preferentially 1.8 cineole, in relative amounts, based on the drug or medicament, in the range of from 0.0001 to 80 wt.-%, especially in the range of from 0.001 to 75 wt.-%, preferentially in the range of from 0.005 to 70 wt.-%, preferably in the range of from 0.01 to 60 wt.-%, more preferably in the range of from 0.05 to 55 wt.-%, even more preferably in the range of from 0.1 to 50 wt.-%.

According to yet another particular embodiment according to this aspect of the invention, it may also be provided that the at least one pharmaceutically acceptable and/or physiologically harmless excipient (carrier) is miscible with cineole, preferentially 1,8-cineole, and/or soluble therein, preferentially wherein the at least one pharmaceutically acceptable and/or physiologically harmless excipient (carrier) is in liquid or solid aggregate state at 20° C. and at atmospheric pressure and/or preferentially wherein the at least one pharmaceutically acceptable and/or physiologically harmless excipient (carrier) is selected from the group of fatty acid esters, preferably triglycerides of fatty acids, more preferably medium-chain triglycerides (MCT), even more preferably triglycerides of C₆-C₁₂-fatty acids.

In this regard, the cineole, preferentially 1,8-cineole, especially the drug or medicament, can be administered in pharmaceutically effective or therapeutically effective amounts.

Especially, the cineole, preferentially 1,8-cineole, especially the drug or medicament, may be prepared for administration in pharmaceutically effective or therapeutically effective amounts.

In addition, the cineole, preferentially 1,8-cineole, especially the drug or medicine, can be applied systemically.

According to the invention, in this context the cineole, preferentially 1,8-cineole, especially the drug or medicament, or the composition, can be used or applied together with at least one further active ingredient, especially wherein the further active ingredient is selected from the group of (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms positively influencing the intestinal flora; as well as combinations thereof.

For further details on this aspect of the invention, reference may be made to the foregoing discussion of the other aspects of the invention, which discussion applies equally to the present aspect of the invention.

Again further object of the present invention—according to a sixth aspect of the present invention is moreover the composition according to the invention, especially pharmaceutical composition, for (use in) reducing pathogenic germs in the human intestine, especially in the human intestinal flora, and/or for (use in) reducing the colonization of the human intestine, especially the human intestinal flora, with pathogenic germs, preferentially for the purpose of preventing, reducing or curing inflammatory diseases of the human body and/or preferentially for the purpose of prophylactic and/or therapeutic treatment of inflammatory diseases of the human body, wherein the composition comprises cineole, preferentially 1,8-cineole, especially together with at least one pharmaceutically acceptable and/or physiologically harmless excipient (carrier).

In this context, the present invention according to this aspect also relates to the composition according to the invention, preferentially pharmaceutical composition, especially as previously defined, for (use in the) prophylactic and/or therapeutic treatment of dysbiosis and/or of malcolonization of the human intestine, especially of the human intestinal flora, with pathogenic germs, wherein the composition contains cineole, preferentially 1,8-cineole, especially together with at least one pharmaceutically acceptable and/or physiologically harmless excipient (carrier).

The composition may contain the cineole, preferentially 1,8-cineole, as the sole active ingredient, especially as the sole pharmaceutical active ingredient.

In addition, the composition may contain the cineole, preferentially 1,8-cineole, as a pure substance, preferentially with a purity of at least 95 wt.-%, especially at least 96 wt.-%, preferably at least 97 wt.-%, more preferably at least 98 wt.-%, even more preferably at least 99 wt.-%, most preferably at least 99.5 wt.-%, based on the cineole, preferentially 1,8-cineole, and/or preferably free of any other terpenes.

The composition according to the invention may contain the cineole, preferentially 1,8-cineole, free of any other terpenes.

Especially, the composition may contain no other terpene. Similarly, the composition may be free of terpenes other than cineole, preferentially 1,8-cineole.

According to the invention, it is particularly intended that the composition contains the cineole, preferentially 1,8-cineole, in effective, especially pharmaceutically and/or therapeutically effective, amounts.

In this regard, the composition may contain the cineole, preferentially 1,8-cineole, in relative amounts, based on the composition, in the range of from 0.0001 to 80 wt.-%, especially in the range of from 0.001 to 75 wt.-%, preferentially in the range of from 0.005 to 70 wt.-%, preferably in the range of from 0.01 to 60 wt.-%, more preferably in the range of from 0.05 to 55 wt.-%, even more preferably in the range of from 0.1 to 50 wt.-%.

Especially, the at least one pharmaceutically acceptable and/or physiologically harmless excipient (carrier) may be miscible with and/or soluble in cineole, preferentially 1,8-cineole, preferentially wherein the at least one pharmaceutically acceptable and/or physiologically harmless excipient (carrier) is in liquid or solid aggregate state at 20° C. and at atmospheric pressure.

In this respect, the at least one pharmaceutically acceptable and/or physiologically harmless excipient (carrier) may be selected from the group of fatty acid esters, preferably triglycerides of fatty acids, more preferably medium-chain triglycerides (MCT), even more preferably triglycerides of C₆-C₁₂-fatty acids.

Especially, the cineole, preferentially 1,8-cineole, especially the composition, may be adapted for administration in pharmaceutically effective or therapeutically effective amounts. Especially, the cineole, preferentially 1,8-cineole, especially the composition, may be prepared for administration in pharmaceutically effective or therapeutically effective amounts. Especially, the cineole, preferentially 1,8-cineole, especially the drug or medicament or the composition, may be administered systemically.

Also in this respect, the cineole, preferentially 1,8-cineole, especially the composition, can be used or applied together with at least one further active ingredient, especially wherein the further active ingredient is selected from the group of (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms positively influencing the intestinal flora; as well as combinations thereof.

For further details on this aspect of the invention, reference can be made to the explanations on the other aspects of the invention, whereby these explanations apply equally to the present aspect of the invention.

Finally, it is equally an object of the present invention—according to a seventh aspect—of the present invention—to provide the pharmaceutical combination for (use in) reducing pathogenic germs in the human intestine, especially in the human intestinal flora, and/or for (use in) reducing the colonization of the human intestine, especially the human intestinal flora, with pathogenic germs, preferentially for the purpose of preventing, reducing or curing inflammatory diseases of the human body and/or preferentially for the purpose of prophylactic and/or therapeutic treatment of inflammatory diseases of the human body, wherein the pharmaceutical combination comprises at least the following components (A) and (B):
(A) a cineole, preferentially 1,8-cineole; as well as
(B) at least one further active ingredient selected from the group of (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms which positively influence the intestinal flora; as well as combinations thereof.

According to the present aspect, the present invention also relates to the pharmaceutical combination, especially a pharmaceutical combination as defined hereinabove, for (use in the) prophylactic and/or therapeutic treatment of dysbiosis and/or of malcolonization of the human intestine, especially of the human intestinal flora, with pathogenic germs, wherein the pharmaceutical combination comprises at least the following components (A) and (B):
(A) a cineole, preferentially 1,8-cineole; as well as
(B) at least one further active ingredient selected from the group of (i) anti-inflammatory active ingredients (anti-inflammatories), especially non-steroidal anti-inflammatory drugs (NSAIDs) or steroidal anti-inflammatory drugs, preferentially steroidal anti-inflammatory drugs, preferably corticosteroids; (ii) antibiotics; (iii) prebiotics; and (iv) microorganisms which positively influence the intestinal flora; as well as combinations thereof.

Also within the scope of this aspect of the invention, it is particularly intended that the active ingredient of component (A) or the cineole, preferentially 1,8-cineole, is administered in pharmaceutically effective or therapeutically effective amounts or that the cineole, preferentially 1,8-cineole, is prepared for administration in pharmaceutically effective or therapeutically effective amounts. This also applies in a corresponding manner to the active ingredient according to component (B).

Especially, the active ingredient of component (A) or the cineole, preferentially 1,8-cineole, can be applied systemically. The other active ingredient according to component (B) can especially also be applied systemically.

According to a particular embodiment of this aspect of the invention, it may especially be provided that the components (A) and (B) are present and/or administered separately from one another, especially spatially separately from one another, but functionally coherent and/or functionally associated with one another.

Furthermore, according to another particular embodiment of this aspect of the invention, it may especially be provided that the pharmaceutical combination is in the form of a kit (kit-of-parts), especially as a kit of components (A) and (B).

Especially, components (A) and (B) may be present and/or prepared and/or administered as a kit (kit-of-parts).

In the context of the present invention, a kit or kit-of-parts is understood to be especially a unit or arrangement or combination of the two components (A) and (B), wherein the two components (A) and (B) are present separated and/or separately, especially spatially separated and/or spatially separately, but are provided or administered as components that functionally belong together or are functionally associated with one another.

For further details on this aspect of the invention, reference can be made to the explanations on the other aspects of the invention, whereby these explanations apply equally to the present aspect of the invention.

Further advantageous properties, aspects and features of the present invention will also be apparent from the following description of embodiments shown in the figures, as still indicated in detail below. It shows:

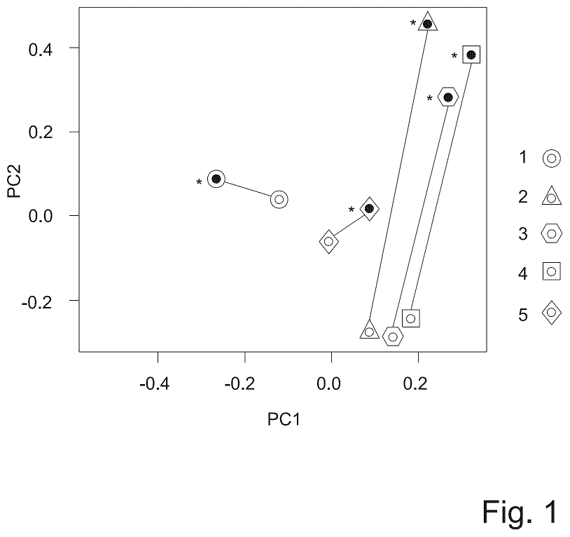

FIG. 1 A graphical illustrations of a principal coordinates analysis (PCoA) of examined stool samples from five patients (1-5) with polyposis nasi, before (without labeling, unfilled circle symbols) and after 2-week ingestion of 1,8-cineole (labeled with “*”, filled circle symbols);

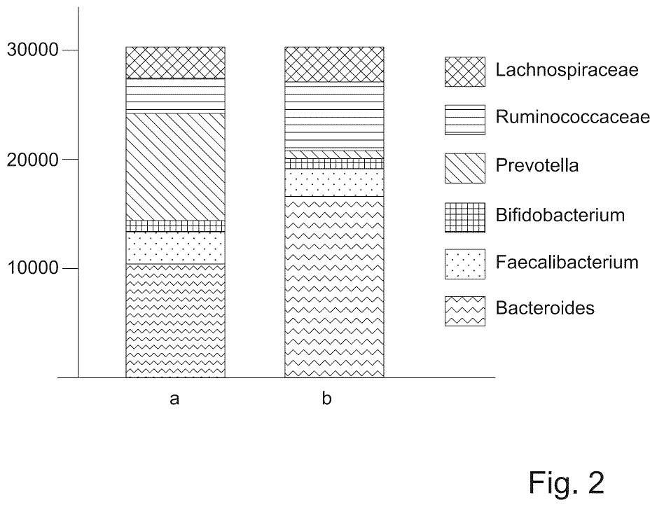

FIG. 2 a graphical illustration in the form of a bar chart of the influence of 1,8-cineole on the intestinal microbiome or intestine flora, respectively, of patients with polyposis nasi; with respect to the X-axis, the designation “a” shows the situation or composition of the intestinal microbiome before administration of cineole and the designation “b” shows the situation in this regard after 2 weeks of taking cineole; furthermore, the Y-axis shows the number of sequencing reads performed;

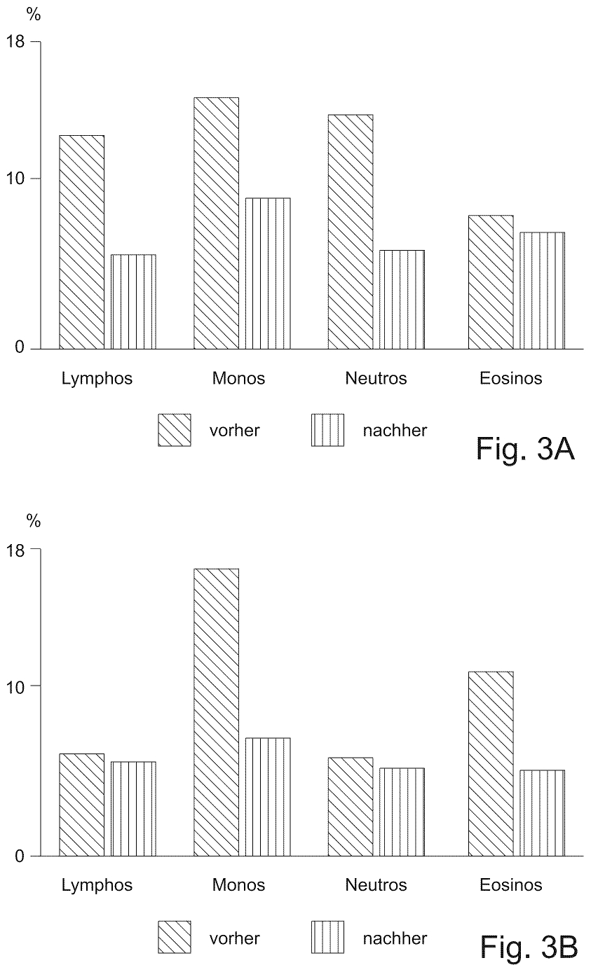

FIG. 3A/B a respective graphical illustrations in the form of bar charts, showing that the ingestion of 1,8-cineole causes a significant decrease in the cellular complexes of various leukocytes [with “lymphos”=lymphocytes; “monos”=monocytes; “neutros”=neutrophils; “eosinos”=eosinophils in the blood of patients with polyposis nasi; shown in each case is the situation before (“vorher”) and after 2-week administration of 1,8-cineole (“nachher”); FIG. 3A refers to data n1, and FIG. 3B refers to data n2, see also following comments)];

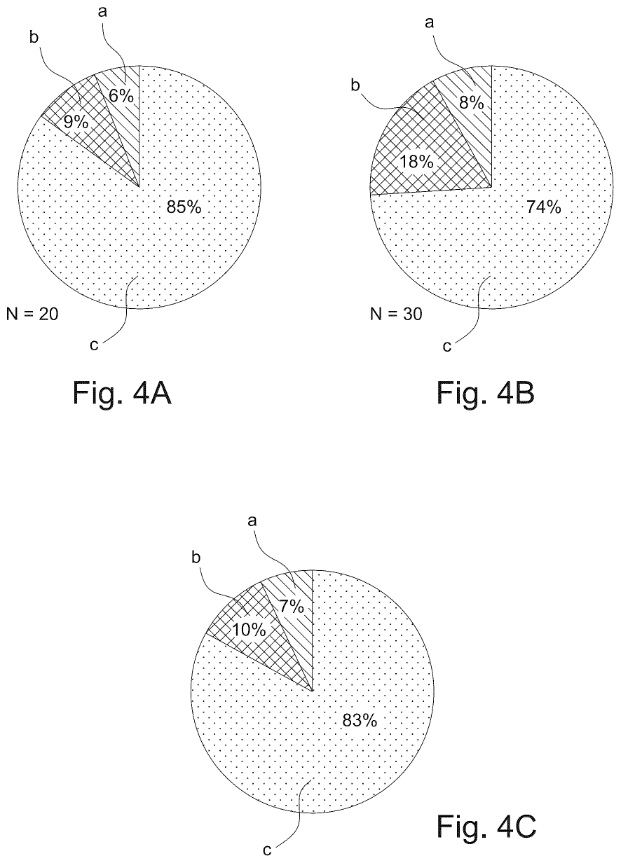

FIG. 4A/B/Ca respective graphical illustrations based on pie charts showing an overall decrease in the population of so-called “classical” monocytes and a clear increase of so-called “intermediate” monocytes in the blood of examined patients with polyposis nasi (FIG. 4B) compared to healthy donors (FIG. 4A), according to which it is further shown that after ingestion of 1,8-cineole over a period of two weeks there is a clear backward shift with respect to the distribution of the respective monocytes (FIG. 4C), which is comparable to the distribution pattern of healthy donors; in this context, the pie chart according to FIG. 4A thus shows the situation for healthy donors, the pie chart according to FIG. 4B thus shows the situation for patients with polyposis nasi and the pie chart according to FIG. 4C shows the situation for patients with polyposis nasi after taking 1,8-cineole for a period of two weeks (with a=“non-classical” monocytes, b=“intermediate” monocytes and c=“classical” monocytes);

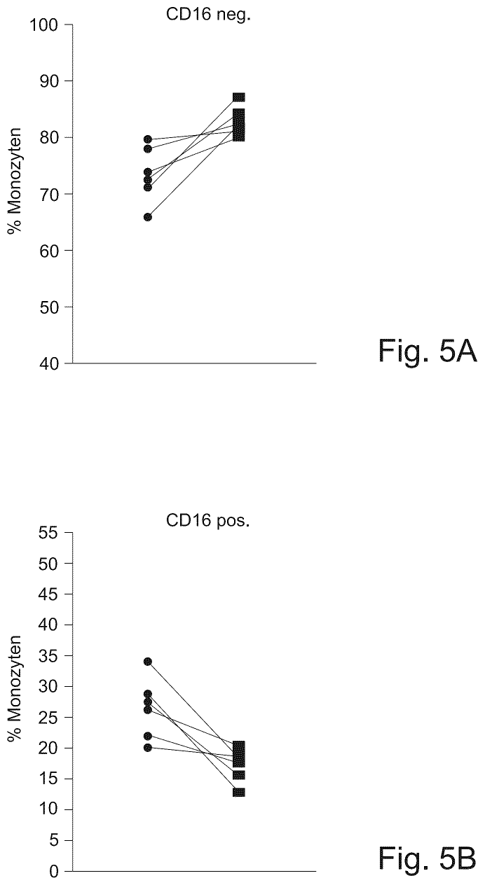

FIG. 5A/B a respective graphical illustrations of monocyte distribution under the influence of 2-week therapy with 1,8-cineole or before and after 2-week therapy with 1,8-cineole, respectively; the studies show a strong response to the intake of 1,8-cineole, with a clear increase in CD16 “classical” monocytes (“CD16 neg.”; FIG. 5A”) and at the same time a clear decrease of “intermediate” monocytes (“CD16 pos.”; FIG. 5B”) after therapy with 1,8-cineole [in FIG. 5A/B the respective left circle representation shows the situation before performing the cineole treatment and the respective right box representation shows the situation after performed cineole therapy].

For further details on the embodiments and exemplary embodiment shown in the figure illustrations, reference can be made to the above explanations and to the following supplementary explanations on the embodiment examples, which apply accordingly with respect to the figure illustrations, in order to avoid unnecessary repetition.

Further embodiments, modifications and variations as well as advantages of the present invention are readily apparent and realizable to those skilled in the art upon reading the description without departing from the scope of the present invention.

The following embodiments are merely illustrative of the present invention without, however, limiting the present invention thereto.

### EXAMPLES

### Example 1: Anti-Inflammatory Effect of 1,8-cineole on the Intestinal Microbiome as Well as Antidysbiotic or Antipathogenic Effect of 1,8-Cineole with Respect to Pathogenic Intestinal Germs

The human microbiome, and especially the intestinal microbiome, has fundamental regulatory influence on inflammatory processes and associated immune regulation.

It can be shown that 1,8-cineole administered as a small intestine soluble preparation (Soledum® forte, content of cineole: 200 mg) has a direct local influence on the microbiome of the intestine, with far-reaching effects on systemic and local inflammatory processes in the human body also being observed.

As part of the studies, the stool samples of patients with polyposis nasi are analyzed under the influence of 1,8-cineole. For this purpose, a stool sample is first taken before starting the administration of 1,8-cineole and then again after 2 weeks of taking 1,8-cineole 3 times a day (Soledum® forte, content of 1,8-cineole: 200 mg).

Stool Stabilizer (company: Stratec Molecular) is added to the stool samples and initially stored at a temperature of −20° C. until further processing. Subsequent DNA extraction is performed using the PSP Spin Stool DNA Plus kit according to the manufacturer's instructions (Stratec Molecular). Finally, the isolated DNA is used for microbiome sequencing (TG MiSeq Reagent Kit v3 & NEBNext® Library Quant Kit for Illumina) and analyzed.

The results are illustrated in FIG. 1 in the form of principal coordinate analysis (PCoA) of the examined stool samples of the patients (1 to 5) with polyposis nasi before (without labeling, unfilled circle symbols) and after 2-week ingestion of 1,8-cineole (marked with “*”, filled circle symbols).

The studies show in the analyzed patient samples a clear shift of the so-called beta diversity based on the principal coordinates analysis (PCoA), i.e. the heterogeneity or, so to speak, the dissimilarity of the measured samples before and after the ingestion of 1,8-cineole, which means that under the influence of 1,8-cineole there is a corresponding change.

Following this initial evaluation regarding existing alterations or changes in the intestinal microbiome of patients with polyposis nasi after 2-week ingestion of 1,8-cineole, a detailed evaluation of the microbiome sequencing performed will be performed regarding the bacterial genera identified.

The relevant studies show a clear shift in the composition of the intestinal microbiome of patients with polyposis nasi after taking 1,8-cineole for 2 weeks. FIG. 2 illustrates the influence of 1,8-cineole on the intestinal microbiome of patients with polyposis nasi (where “a” refers to the situation or composition of the intestinal microbiome before administration of cineole and “b” refers to the situation after 2-week intake of cineole; furthermore, the Y-axis shows the number of sequencing reads performed).

In this context, FIG. 2 thus illustrates the clear shift of various bacterial genera found according to the invention: Especially, the analyses of the microbiome show that the bacterial genus Prevotella is very prominently present as a pathogenic or pro-inflammatory germ in the intestines of the patients with polyposis nasi studied and is significantly reduced after 2-week ingestion of 1,8-cineole, with even an almost complete disappearance being observed in this respect.

The genus Prevotella generally includes about forty different species, which often cause various inflammatory processes of the oral cavity (Prevotella bivia, Prevotella disiens) or respiratory tract (Prevotella melaninogenica, Prevotella intermedia).

In addition, a clear change in the occurrence of germs, for example in the very common genus Bacteroides, which is non-pathogenic and, especially, beneficial to health and has overall positive effects for humans, can be observed, namely to the effect that these are significantly more abundant after therapy with 1,8-cineole. In the case of other families or genera belonging to the normal human intestinal flora, such as Lachnospiraceae, Bifidobacterium or Faecalibacterium, certain changes or shifts with a tendency to increase after implementation of the therapy can also be observed.

Further evaluations of the microbiome sequencing performed also lead to the identification of a significant increase in bacteria of the family Ruminococcacea or the genus Ruminococcus as a result of ingestion of 1,8-cineole, wherein such bacteria being known to produce anti-inflammatory short-chain fatty acids such as butyrate.

The studies thus show the positive effects of 1,8-cineole on the composition of the human intestinal microbiome or intestinal flora with the reduction of inflammatory or negative germs and the increase of anti-inflammatory or positive germs, which is accompanied by an influence also on total body or systemic inflammatory processes.

### Example 2: Anti-Inflammatory Effect of 1,8-cineole in Relation to Immune Cells in Peripheral Blood

As explained above, 1,8-cineole firstly has a positive effect at the primary site of action, namely the human intestine, on the intestinal epithelium with the immune cells present there, and especially on the basis of the reduction of pathogenic germs in the human intestinal microbiome, so that their negative influence on the intestinal epithelium is reduced or prevented (cf. preceding example of embodiment). This in turn leads to an improvement in the immune status in this regard (i.e. induction of active or primary anti-inflammatory mediation). This is documented by the following experiment.

Presented are studies with regard to the regulation of circulating blood cells/immune cells in patients with polyposis nasi under the influence of 1,8-cineole.

Analysis by fluorescence microscopy showed a marked increase in the complex formation of circulating platelets (CD41) with different immune cells (DAPI) in patients with polyposis nasi and also suggested a regulation of immune functions such as the expression levels of the cytokine TNFa.

Further, these cellular complexes will be specified in more detail by flow cytometer with respect to different leukocyte populations and with respect to the influence of 1,8-cineole.

Here it is shown that these cellular complexes occur in the blood of patients with polyposis nasi particularly in connection with monocytes and that after taking 1,8-cineole (cf. above) over a period of two weeks a massive reduction of these monocyte complexes can be observed, as shown in the following tables and in FIG. 3A/3B [with “lymphos”=lymphocytes; “monos”=monocytes; “neutros”=neutrophils; “eosinos”=eosinophils in the blood of patients with polyposis nasi; shown is the situation before (“vorher”) and after 2-week administration of 1,8-cineole (“nachher”); FIG. 3A refers to data n1 according to Table 1, and FIG. 3B refers to data n2 according to Table 2].

TABLE 1

| n1 | before | after |
|---|---|---|
| Lymphos | 12.4% | 5.54% |
| Monos | 14.7% | 8.79% |
| Neutros | 13.9% | 5.02% |
| Eosinos | 7.3% | 5.92% |

TABLE 2

| n1 | before | after |
|---|---|---|
| Lymphos | 5.4% | 5.12% |
| Monos | 16.8% | 6.28% |
| Neutros | 5.37% | 5.09% |
| Eosinos | 10.7% | 4.8% |

The monocytes circulating in the blood can be divided into the three different subtypes, namely “classical”, “intermediate” and “non-classical”, on the basis of their surface proteins CD14 and CD16 by flow cytometry. In flow cytometry, cells are analyzed for cell size, granularity and fluorescent labeling of specific epitopes by passing specific lasers. This allows different cell populations to be differentiated and characterized. While “classical” monocytes differentiate into anti-inflammatory macrophages after immigration into inflammatory tissue, “non-classical” monocytes become immunosuppressive macrophages that do not stop the inflammatory processes and thus maintain the associated disease pattern.

The studies show a decrease in the population of “classical” monocytes and a significant increase in the population of “intermediate” monocytes in the blood of the examined patients with polyposis nasi compared to healthy donors, thus indicating a possibly increased infiltration of the inflammatory polyp tissue by immunosuppressive macrophages and a resulting persistent inflammatory progression of the nasal polyps. In this regard, reference can also be made to FIG. 4 A/B/C.

In this context, FIG. 4 A/B/C shows the found decrease in the population of “classical” monocytes and the significant increase of “intermediate” monocytes in the blood of the studied patients with polyposis nasi in comparison with healthy donors.

After 2-week intake of 1,8-cineole, monocyte populations in the blood of patients with polyposis nasi are again examined by flow cytometer, and a marked normalization or reversion of the distribution of monocyte subpopulations is observed, comparable to the distribution pattern in healthy individuals or donors.

FIG. 4 A/B/C shows an overall decrease in the population of “classical” monocytes and a significant increase in “intermediate” monocytes in the blood of patients with polyposis nasi (FIG. 4B) compared to healthy donors (FIG. 4A). In addition, it is shown that after ingestion of 1,8-cineole over a period of two weeks, a clear normalization or reversion results with respect to the distribution of the respective monocytes (FIG. 4C), which is comparable to the distribution pattern of healthy donors. The pie chart according to FIG. 4A shows the situation for healthy donors, the pie chart according to FIG. 4B the situation for patients with polyposis nasi and the pie chart according to FIG. 4C the situation for patients with polyposis nasi after therapy or intake of 1,8-cineole for a period of two weeks (with a=“non-classical” monocytes, b=“intermediate” monocytes and c=“classical” monocytes).

These observations are now comprehensively analyzed in additional patients individually by flow cytometer, especially under the influence of 1,8-cineole. The investigations confirm a strong response of the circulating immune cells to the intake of 1,8-cineole in the examined patients, accompanied by a restoration of the monocyte distribution, comparable to the healthy situation. In this regard, reference can also be made to FIG. 5A/B.

FIG. 5A/B the monocyte distribution under the influence of 2-week therapy with 1,8-cineole or before and after 2-week therapy with 1,8-cineole; the studies show a strong response to the intake of 1,8-cineole, with a significant increase in CD16 “classical” monocytes (“CD16 neg.”; FIG. 5A”) and at the same time a clear decrease of “intermediate” monocytes (“CD16 pos.”; FIG. 5B”) can be observed after therapy with 1,8-cineole (in FIG. 5A/B the respective left circle representation shows the situation before cineole treatment and the respective right box representation shows the situation after cineole treatment).

FIG. 5 A/B thus shows the monocyte distribution under the influence of a 2-week therapy with 1,8-cineole, showing a strong response to the intake of 1,8-cineole. In the patients studied, there is a marked increase in CD16 “classical” monocytes (FIG. 5A) and at the same time a marked decrease in “intermediate” monocytes (FIG. 5B).

### Example 3: Patient Perceptions and Clinical Applications

As equally explained above, 1,8-cineole not only has a positive effect on the intestinal epithelium with the immune cells present there at the primary site of action, namely the human intestine, but also with regard to improving the immune status in this respect (i.e. induction of active or primary anti-inflammatory mediation). This in turn also leads to a systemically relevant improvement of inflammatory processes or parameters in the human body when applied clinically. This can be demonstrated by the therapy of a wide variety of inflammatory diseases of the human body (which is an indication of organ-unspecific or regio-unspecific and thus systemically universal anti-inflammatory activity).

In addition to the molecular biological investigations, the individual perception and quality of life of the patients with regard to the 2-week intake of 1,8-cineole is also surveyed. Of the polyposis patients consulted, the response to the 2-week intake of 1,8-cineole was predominantly positive.

In 38 patients (23 men/15 women between 28 and 72 years, average age 54 years) each with polyposis nasi a therapy with 1,8-cineole was performed (3 times 200 mg 1,8-cineole in the form of capsules “Soledum® forte”).

The treated patients report significantly improved secretion drainage and correspondingly improved nasal breathing. In one case, the patient was diagnosed with relatively small nasal polyps localized directly on the olfactory groove. In this patient, after 2 weeks of taking 1,8-cineole, a significant decrease in the polyps is noted, with a marked improvement in olfactory sensation as well.

### Example 4: 1,8-Cineole in Clinical Use

In addition, the effect of 1,8-cineole is further investigated on the basis of the following areas of application, with 200 mg of 1,8-cineole being administered 3 times a day for the period indicated in each case (Soledum® forte):
1. Chronic polypous sinusitis
2. Chronic recurrent catarrhal sinusitis
3. Chronic polyposis otitis media without cholesteatoma
4. Chronic polyposis otitis media with cholesteatoma
Ad 1. After 2-week administration (preoperatively), reduction of the accompanying symptoms of the polyps, such as anterior and posterior watery rhinorrhea. In individual cases also improvement of olfactory performance; postoperative positive influence on wound healing and in combination with a nasal steroid reduction of recurrences and systemic cortisone administration;
Ad 2. After 2-week administration (preoperatively), decrease in concomitant symptoms of polyps, such as anterior and posterior watery rhinorrhea; significant improvement in olfactory performance; postoperative positive influence on wound healing, reduction of nasal steroids;
Ad 3. After 2-week administration (preoperatively) in previously refractory otorrhea (local and systemic antibiotics), decrease in otorrhea, allowing surgery; in condition after tympanoplasty, renewed otorrhea with recurrent perforation; after 4-week administration, decrease in otorrhea and spontaneous tympanic membrane closure;
Ad 4. After 2-week administration (preoperatively) in previously refractory otorrhea (local and systemic antibiotics), decrease in concomitant otorrhea, allowing surgery; no reduction in cholesteatom masses, but positive effect on inflamed mucosa of middle ear and mastoid; in condition after tympanoplasty, renewed otorrhea with recurrent perforation. After 4 weeks of administration, decrease of otorrhea and spontaneous tympanic membrane closure.

### Example 5: 1,8-Cineole in Further Clinical Use

Patients with persistent bronchial asthma who are treated with a combination therapy of inhaled glucocorticoid and inhaled long-acting beta-2-sympathomimetic as well as theophylline perorally receive 1,8-cineole (Soledum® forte capsules) 4 times 200 mg/die perorally for one week. A slight to moderate improvement in lung function can be achieved after only one week of therapy with the aforementioned dose. After twelve weeks of continuous therapy, persistent bronchial asthma has stabilized to such an extent that inhaled glucocorticoid requirements can be reduced by more than 50% and, in some of the patients treated, glucocorticoids can even be temporarily discontinued altogether. In some patients, the need for betamimetics can also be significantly reduced. Therapy with 1,8-cineole is well tolerated without side effects. The experimental results show that 1,8-cineole, due to its anti-inflammatory effect, can significantly increase the efficacy of inhaled airway therapeutics and thus significantly reduce their dose requirement.

Overall, based on the surprisingly discovered findings of the applicant, cineole, preferentially 1,8-cineole, is thus efficiently suitable for use in reducing pathogenic germs in the human intestine or for use in reducing the colonization of the human intestine with pathogenic germs, preferably for the purpose of preventing, reducing or curing inflammatory diseases of the human body or, preferably, for the purpose of prophylactic and/or therapeutic treatment of inflammatory diseases of the human body.

1. An intestinal treatment drug for the treatment of a dysbiosis including a malcolonization of the human intestine with pathogenic germs,
wherein the intestinal treatment drug comprises a composition comprising, as the sole active ingredient and in the absence of any further active ingredients, a 1,8-cineole together with at least one physiologically acceptable excipient, wherein the physiologically acceptable excipient is miscible with the 1,8-cineole and is selected from the group consisting of triglycerides of C₆-C₁₂-fatty acids,
wherein the composition comprises the 1,8-cineole in relative amounts, based on the composition, in the range of from 0.01 to 60 wt.-% and
wherein the composition comprising the 1,8-cineole is formulated as a systemic dosage form which is resistant to gastric juice but soluble in the small intestine;
wherein the intestinal treatment drug effects, when administered in a therapeutically effective amount to a patient suffering from a dysbiosis including a malcolonization of the human intestine with pathogenic germs, reducing pathogenic germs in the human intestine and reducing the colonization of the human intestine with pathogenic germs and additionally effects increasing the colonization of the human intestine with at least one of health-promoting and non-pathogenic germs, wherein the pathogenic germs are selected from the group consisting of inflammation-causing bacteria, inflammation-inducing bacteria and bacteria associated with inflammations in the human body and combinations thereof and wherein the health-promoting and non-pathogenic germs are selected from the group consisting of inflammation-reducing germs, anti-inflammatory germs and inflammation-inhibiting germs and combinations thereof.

2. The intestinal treatment drug according to claim 1,
wherein the 1,8-cineole is present as a pure substance having a purity degree of at least 95 wt.-%, based on the 1,8-cineole, and is absent from any other terpenes.

3. The intestinal treatment drug according to claim 1,
wherein the 1,8-cineole is present as a pure substance having a purity degree of at least 99 wt.-%, based on the 1,8-cineole, and is absent from any other terpenes.

4. The intestinal treatment drug according to claim 1,
wherein the composition comprises the 1,8-cineole in relative amounts, based on the composition, in the range of from 0.05 to 55 wt.-%.

5. The intestinal treatment drug according to claim 1,
wherein the composition comprises the 1,8-cineole in relative amounts, based on the composition, in the range of from 0.1 to 50 wt.-%.

6. The intestinal treatment drug according to claim 1,
wherein the intestinal treatment drug is in the form of a capsule, dragee, pill or tablet.

7. A composition for the treatment of a dysbiosis including a malcolonization of the human intestine with pathogenic germs,
wherein the composition comprises, as the sole active ingredient and in the absence of any further active ingredients, a 1,8-cineole together with at least one physiologically acceptable excipient, wherein the physiologically acceptable excipient is miscible with the 1,8-cineole and is selected from the group consisting of triglycerides of C₆-C₁₂-fatty acids,
wherein the composition comprises the 1,8-cineole in relative amounts, based on the composition, in the range of from 0.01 to 60 wt.-% and
wherein the composition comprising the 1,8-cineole is formulated as a systemic dosage form which is resistant to gastric juice but soluble in the small intestine;
wherein the composition effects, when administered in a therapeutically effective amount to a patient suffering from a dysbiosis including a malcolonization of the human intestine with pathogenic germs, reducing pathogenic germs in the human intestine and reducing the colonization of the human intestine with pathogenic germs and additionally effects increasing the colonization of the human intestine with at least one of health-promoting and non-pathogenic germs, wherein the pathogenic germs are selected from the group consisting of inflammation-causing bacteria, inflammation-inducing bacteria and bacteria associated with inflammations in the human body and combinations thereof and wherein the health-promoting and non-pathogenic germs are selected from the group consisting of inflammation-reducing germs, anti-inflammatory germs and inflammation-inhibiting germs and combinations thereof.
